(19) United States
(12) Patent Application Publication
(10) Pub. No.: US 2026/0306080 A1
(43) Pub. Date: Oct. 8, 2026

(54) SAMPLE COLLECTION DEVICE AND ASSOCIATED METHODS

(71) Applicant: COPAN ITALIA S.p.A., Brescia (IT)

(72) Inventors: Giorgio MARTELLO, BRESCIA (IT); Matteo ZANI, BRESCIA (IT)

(21) Appl. No.: 19/474,797

(22) PCT Filed: Apr. 11, 2024
(86) PCT No.: PCT/IB2024/053527

§ 371 Date: Oct. 13, 2025

(30) Foreign Application Priority Data
Apr. 13, 2023 (IT) 102023000007119

(51) Int. Cl. A61B 10/02; A61B 10/00; A61B 90/00

(52) U.S. Cl. CPC A61B 10/02; A61B 10/0051; A61B 90/08; A61B 2010/009; A61B 2090/0807

(57) ABSTRACT

Device (1) for the collection and the transfer of samples of biological, microbiological or chemical material, comprising: a body (2), preferably elongated and extending at least partially along a main development direction (X) substantially axial, comprising at least one gripping portion (2i) destined to be grasped by a user and at least one ending portion (2e); a collecting portion (3), preferably arranged in correspondence of said ending portion (2e), the collecting portion being configured for collecting and/or absorbing and/or retaining and/or releasing a quantity of a sample of biological, microbiological or chemical material; at least one wireless identification electronic device (10), configured for receiving and/or transmitting and/or storing electronic data on a wireless communication channel, the wireless identification electronic device (10) being arranged, at least partially, within said body (2) and/or within said collecting portion (3).

### FIELD OF THE TECHNIQUE

The present disclosure relates to the field of devices suitable for the collection of substances, and in particular of samples, for example of biological, microbiological or chemical type.

### KNOWN ART

The so-called “swabs” are devices for the collection of samples provided with an elongated body, preferably extending along a predefined and main extension axis, to which it is connected a collecting portion which, in the known art, includes fibrous material preferably with typically hydrophilic properties.

These devices are typically used to allow the collection of samples of biological or microbiological material, for example—but not limited to—from cavities of the body of a subject who may be human or animal, or of chemical type.

In some cases, the above-mentioned devices may take on connotations of nasopharyngeal swabs. The nasopharyngeal swabs are typically introduced in the nasal cavity through the opening of a naris, and are pushed in the nasal cavity until typically reaching a mucosa present in the nasopharynx, which is substantially placed in correspondence of the rear portion of the nasal cavity, for collecting biological samples, such as epithelial cells, secretions, etc., of the upper respiratory tracts.

After the collection, the nasopharyngeal swab is extracted from the nose and the collected sample can subsequently be placed for example in a reagent, to determine the presence or absence of determined pathogens, subjected to other typologies of analysis, or put in contact with substances designed to allow its preservation over time and its transport to a laboratory.

In addition, these devices can also be used for the collection of nonbiological substances and from surfaces or cavities that are not part of a subject, for example from roads, walls, vehicles.

The above-mentioned devices are typically destined to allow to preserve, possibly without significant alteration, the collected sample. For this reason they are typically associated with containers, within which at least the collecting portion, and preferably also the elongated body, are contained in order to preserve the collected sample from external agents, which could contaminate it.

The container may also contain a chemical substance or chemical compound, destined to preserve the sample, or to detect the presence of a determined substance in the sample or even to identify the nature of the sample.

On the container can typically be attached tags that allow cataloging the sample collected in the collecting portion of the device contained in the container. In use, on the tags some data is manually written by an operator.

The Applicant notes that any writing errors in the tag can lead to problems in the correct cataloging of the sample collected in the collecting portion. In fact, it is noted that samples are typically kept for a long time inside the container and are often stored together with other similar samples, and this—in case of an error—makes it even more problematic to determine accurately and a posteriori, the actual origin, content and/or date of collection of the sample.

It is also known to attach to containers tags printed in an automatic way or other typologies of markers, but such tags or markers may be altered or damaged over time, so it is possible that information relative to the sample may be altered or lost, with potentially serious consequences for example for the health of patients.

The purpose of the present disclosure is to describe a device for collection of samples that allows to overcome the above-described drawbacks.

### SUMMARY

The object of the present disclosure is now described in some of its salient aspects, which can be combined with each other or with portions of the detailed description and/or claims.

According to the present disclosure it is first of all described a device (1) for the collection and the transfer of samples of biological, microbiological or chemical material, comprising:
a body (2), comprising at least one gripping portion (2i) destined to be grasped by a user and at least one ending portion (2e);
a collecting portion (3), preferably arranged in correspondence of said ending portion (2e), the collecting portion being configured for collecting and/or absorbing and/or retaining and/or releasing a quantity of a sample of biological, microbiological or chemical material;
at least one wireless identification electronic device (10), configured for receiving and/or transmitting and/or storing electronic data on a wireless communication channel, the wireless identification electronic device (10) being arranged, at least partially, within said body (2) and/or within said collecting portion (3).

According to another non-limiting aspect, the device (1) comprises at least one second wireless identification electronic device (10), configured for receiving and/or transmitting and/or storing electronic data on a wireless communication channel, the wireless identification electronic device (10) being arranged, at least partially, within said body (2) and/or within said collecting portion (3).

According to another non-limiting aspect, the device (1) comprises at least:
a first wireless identification electronic device (10) and at least one second wireless identification electronic device (10) arranged at least partially within said first body (2); or
a first wireless identification electronic device (10) arranged at least partially within said first body (2) and at least one second wireless identification electronic device (10) arranged within said collecting portion (3);
a first wireless identification electronic device (10) and at least one second wireless identification electronic device (10) arranged within said collecting portion (3);
a first wireless identification electronic device (10) arranged within said collecting portion (3) and at least one second wireless identification electronic device (10) arranged at least partially within said first body (2).

According to another non-limiting aspect, the body (2) is elongated and extending at least partially along a main development direction (X) substantially axial.

According to another non-limiting aspect, at least one between said body (2) and said collecting portion (3) is realized by moulding, preferably injection moulded and/or overmoulded, in particular moulded by insertion or with core loss, and/or co-moulding.

According to another non-limiting aspect, at least one between said body (2) and said collecting portion (3) is realized by overmoulding on, or at least partially around a, said at least one wireless identification electronic device (10).

According to another non-limiting aspect, said at least one wireless identification electronic device (10) is joined to said body (2) and/or to said collecting portion (3) in said moulding, and preferably in said injection moulding and/or in said overmoulding, in particular by insertion or with core loss, and/or in said co-moulding, being introduced within a mould (200).

According to another non-limiting aspect, said first and said second wireless identification electronic device (10) are joined to said body (2) and/or to said collecting portion (3) in said moulding, and preferably in said injection moulding and/or in said overmoulding, in particular by insertion or with core loss, and/or in said co-moulding, being introduced within a mould (200).

According to another non-limiting aspect, said at least one wireless identification electronic device (10) is joined to said body and/or to said collecting portion (3) by gluing and/or insertion by contrast, optionally carried out after said moulding.

According to another non-limiting aspect, said at least one wireless identification electronic device (10) is irremovably fixed to said body (2) and/or to said collecting portion (3).

According to another non-limiting aspect, said body (2) is substantially rigid.

According to another non-limiting aspect, said body (2) is substantially flexible.

According to another non-limiting aspect, said body (2) is substantially flexible along a direction (X) substantially parallel to said main development direction.

According to another non-limiting aspect, said body (2) is substantially incompressible.

According to another non-limiting aspect, said at least one wireless identification electronic device (10) is positioned and enclosed integrally within said body (2) and/or within said collecting portion (3), and/or wherein said body (2) and/or said collecting portion (3) defines a covering layer (4) of said at least one wireless identification electronic device (10),
optionally said covering layer (4) extending around said at least one wireless identification electronic device (10) in a plurality of radial directions, extending in a direction oblique, preferably orthogonal, to the main development direction (X) of said body (2).

According to another non-limiting aspect, said collecting portion (3) is joined to said body (2) and/or is realized integrally and/or seamlessly with said body (2).

According to another non-limiting aspect, said collecting portion (3) comprises:
a swab (3b) preferably having one among the following properties: hydrophilic, hydrophobic or neutral, preferably comprising a plurality of fibers provided with hydrophilic capacity, and optionally being a flocked swab (3b), and/or with wound fiber, and/or in foam;
a supporting portion (3a) for said swab (3b).

According to another non-limiting aspect, said ending portion (2e) corresponds to said supporting portion (3a) and/or is realized seamlessly on said supporting portion (3a).

According to another non-limiting aspect, said at least one wireless identification electronic device (10) is positioned within said supporting portion (3a) and wherein said covering layer (4) is part of said supporting portion (3a).

According to another non-limiting aspect, said at least one wireless identification electronic device (10) comprises a respective outer surface (10x).

According to another non-limiting aspect, said body (2) presents a respective outer surface (2x).

According to another non-limiting aspect, the outer surface (10x) of said at least one wireless identification electronic device (10) is in substantial contact with the outer surface (2x) of the body (2) or faces on said outer surface (2x) of the body (2) and/or protrudes partially outside said body (2).

According to another non-limiting aspect, said at least one wireless identification electronic device (10) is positioned in substantial correspondence of said ending portion (2e) or in correspondence of said gripping portion (2i).

According to another non-limiting aspect, the device (1) comprises at least one weakening portion (5).

According to another non-limiting aspect, said weakening portion (5) is positioned on said body (2) and being configured for facilitating a separation between at least part of the body (2) and the collecting portion (3).

According to another non-limiting aspect, the body (2) comprises at least one first half-body (7) and one second half-body (8) different one another and selectively couplable, for defining an assembled configuration of the body (2), and selectively separable the one from the other, for defining a disassembled condition of the body (2).

According to another non-limiting aspect, the collecting portion (3) is realized and engaged both on a first end of the first half-body (7) and on a first end of the second half-body (8).

According to another non-limiting aspect, the collecting portion (3) is selectively separable, after a separation of the first half-body (7) from the second half-body (8), for defining two half-collecting portions respectively realized on the first half-body (7) and on the second half-body (8).

According to another non-limiting aspect, said at least one wireless identification electronic device (10) is arranged at least partially in said first half-body (7) or in correspondence of the collecting portion (3), preferably of the supporting portion (3a), joined to said first half-body (7) and/or realized integrally and/or seamlessly with said first half-body (7),
or is arranged at least partially in said second half-body (8) or in correspondence of the collecting portion (3), preferably of the supporting portion (3a), joined to said first half-body (7) and/or realized integrally and/or seamlessly with said first half-body (7).

According to another non-limiting aspect, said at least one wireless identification electronic device (10) is a first wireless identification electronic device.

According to another non-limiting aspect, said first wireless identification electronic device (10) is arranged in correspondence of said first half-body (7) and said second wireless identification electronic device (10) is arranged in correspondence of said second half-body (8), and/or
said first wireless identification electronic device (10) is arranged in correspondence of the half-collecting portion realized on the first half-body (7) and said second wireless identification electronic device (10) is arranged in correspondence of the half-collecting portion realized on the second half-body (8), and/or
said first wireless identification electronic device (10) is arranged in correspondence of the first half-body (7) and said second wireless identification electronic device (10) is arranged in correspondence of the half-collecting portion realized on the second half-body (8).

According to another non-limiting aspect, said weakening portion (5) lies in a position axially translated, along said main development direction (X) of the body (2), with respect to the position assumed by the wireless identification electronic device (10).

According to another non-limiting aspect, said at least one wireless identification electronic device (10) is in substantially three-dimensional shape, preferably having its own main extension axis, substantially parallel to said main development direction (X) of the body (2), and/or wherein said at least one wireless identification electronic device (10) is of passive type, or of active type, or of semi-active type, or of semi-passive type.

According to another non-limiting aspect, said at least one wireless identification electronic device (10) comprises at least one memory configured for storing, in use, tracking data of said sample and/or of the device (1) and/or of analysis method of said sample and/or of collection date of said sample, and/or of transport conditions of said sample.

According to another non-limiting aspect, said at least one wireless identification electronic device (10) is of a read only type, or is configured for operating in read only mode, in such a way that the data written in said memory can be only read, or wherein said at least one wireless identification electronic device (10) is of a read-write type, or is configured for operating in read-write mode, in such a way that the data written in said memory can be read and written.

According to another non-limiting aspect, said at least one wireless identification electronic device (10) is configured for transmitting said electronic data on said wireless communication channel towards a reader device (100), optionally with a power of electromagnetic radiation sufficient to allow the reception of said electronic data at least when said reader device (100) lies within 30 cm, or within 20 cm or within 10 cm from the wireless identification electronic device (10) and/or from the device (1).

According to another non-limiting aspect, the device comprises at least one tag (12) having at least one alphanumeric and/or visual code.

According to another non-limiting aspect, said alphanumeric and/or visual code represents at least part of the data stored in the memory of said at least one wireless identification electronic device (10) and/or is destined, when electronically read, to address a reader device (100) to the retrieval, in particular automated, of said data stored in the memory of said at least one wireless identification electronic device (10) or to the retrieval of an electronic copy of said data stored in the memory of said at least one wireless identification electronic device (10).

According to another non-limiting aspect, said tag (12) is positioned on the body (2) or on a gripping and/or closing element (16) of a container (20) for said device (1), fixed to said body (2) in correspondence of said gripping portion (2i) or of a portion of said body (2) opposed with respect to said ending portion (2e).

According to another non-limiting aspect, said at least one wireless identification electronic device (10) is of heat-resistant type (heat-resistant) and/or is of radiation-resistant type (radiation-resistant) and/or is frost-resistant.

According to another non-limiting aspect, said body (2) and/or said collecting portion (3), preferably said supporting portion (3a), comprises or is realized in at least one among the materials of the following list: polyester and derivatives, for example polybutylene terephthalate (PBT) or polyethylene terephthalate (PET); polyether ether ketone (PEEK); polyphenylenesulfide (PPS); acrylonitrile butadiene styrene (ABS); polyurethane (PU); polyvinylchloride (PVC); polymethylmethacrylate (PMMA); high or low density polyester (HDPE, LDPE); polycarbonate; polystyrene; polyamide, in particular aliphatic polyamide, for example Nylon.

According to another non-limiting aspect, said body (2) and/or said collecting portion (3), preferably said supporting portion (3a), comprises or is realized in a thermoplastic or thermosetting material.

According to another non-limiting aspect, said at least one wireless identification electronic device (10) is of half-duplex type or is configured for operating in half-duplex mode.

According to another non-limiting aspect, said at least one wireless identification electronic device (10) is of full-duplex type, or is configured for operating in full-duplex mode.

According to another non-limiting aspect, said at least one wireless identification electronic device (10) comprises an antenna configured for transmitting radioelectric signals along at least one main radiation direction, and preferably on a main radiation plane in which said radiation is isotropic.

According to another non-limiting aspect, said at least one main radiation direction is substantially inclined with respect to the main development direction (X) of the body (2) and preferably said main radiation plane is substantially inclined with respect to the main development direction (X) of the body (2); optionally, said at least a main radiation direction being substantially orthogonal with respect to the main development direction (X) of the body (2) and preferably said main radiation plane is substantially orthogonal with respect to the main development direction (X) of the body (2).

According to the present disclosure is described a method of manufacturing a device (1) for the collection and the transfer of samples of biological, microbiological or chemical material, the method comprising:
a step of manufacturing (1000) a body (2), comprising at least one gripping portion (2i) destined to be grasped by a user and at least one ending portion (2e);
a step of manufacturing (1001) a collecting portion (3) configured for collecting and/or absorbing and/or retaining and/or releasing a quantity of a sample of biological, microbiological or chemical material, the step of manufacturing (1001) comprising preferably an arrangement of the collecting portion (3) in correspondence of said ending portion (2e),
a step of positioning (1002) at least one wireless identification electronic device (10) configured for receiving and/or transmitting and/or storing electronic data on a wireless communication channel, the step of positioning (1002) comprising an arrangement of said at least one wireless identification electronic device (10) at least partially within said body (2) and/or within said collecting portion (3).

According to another non-limiting aspect, the method comprises a step of positioning (1002) of at least one second wireless identification electronic device (10) configured for receiving and/or transmitting and/or storing electronic data on a wireless communication channel, the step of positioning (1002) comprising an arrangement of said at least one wireless identification electronic device (10) at least partially within said body (2) and/or within said collecting portion (3).

According to another non-limiting aspect, the step of positioning (1002) comprises:
an arrangement of a first wireless identification electronic device (10) and of at least one second wireless identification electronic device (10) at least partially within said first body (2); or
an arrangement of a first wireless identification electronic device (10) at least partially within said first body (2) and an arrangement of at least one second wireless identification electronic device (10) within said collecting portion (3);
an arrangement of a first wireless identification electronic device (10) and of at least one second wireless identification electronic device (10) within said collecting portion (3);
an arrangement of a first wireless identification electronic device (10) within said collecting portion (3) and of at least one second wireless identification electronic device (10) at least partially within said first body (2).

According to another non-limiting aspect, the step of manufacturing the body (2) is the step of manufacturing a body preferably elongated and extending at least partially along a main development direction (X) substantially axial.

According to another non-limiting aspect, the step of manufacturing (1000) the body (2) and/or the step of manufacturing (1001) a collecting portion (3) comprises a step of moulding, preferably a step of injection moulding and/or of overmoulding, in particular moulding by insertion or with core loss, and/or co-moulding, of said body (2) and/or said collecting portion (3).

According to another non-limiting aspect, the step of manufacturing (1000) the body (2) and/or the step of manufacturing (1001) the collection manufacturing (3) comprises a step of overmoulding of said body (2) and/or of said collecting portion (3) on, or at least partially around, said at least one wireless identification electronic device (10).

According to another non-limiting aspect, said at least one wireless identification electronic device (10) is joined to said body (2) and/or to said collecting portion (3) in said step of moulding, and preferably in said step of injection moulding and/or in said step of overmoulding, in particular of moulding by insertion or with core loss, and/or co-moulding, being introduced within a mould (200).

According to another non-limiting aspect, said first and said second wireless identification electronic device (10) are joined to said body (2) and/or to said collecting portion (3) in said step of moulding, and preferably in said step of injection moulding and/or in said step of overmoulding, in particular of moulding by insertion or with core loss, and/or co-moulding, being introduced within a mould (200).

According to another non-limiting aspect, the step of manufacturing (1000) the body (2) and/or the step of manufacturing (1001) a collecting portion (3) comprises a step of moulding, preferably a step of injection moulding and/or of moulding by insertion and/or co-moulding, of said body (2) and/or said collecting portion (3), and said at least one wireless identification electronic device (10) is joined to said body (2) and/or to said collecting portion (3) by gluing and/or insertion by contrast, optionally carried out after said moulding.

According to another non-limiting aspect, the assembly realized by the steps of manufacturing (1000, 1001) the body (2) and/or of the collecting portion (3) and by the step of positioning (1002) determining an immovable fastening of said at least one wireless identification electronic device (10) to said body (2) and/or to said collecting portion (3).

According to another non-limiting aspect, the step of positioning (1002) comprises a positioning of said at least one wireless identification electronic device (10) in a position such that, after the step of manufacturing (1000) the body (2) and/or after the step of manufacturing (1001) the collecting portion (3), said at least one wireless identification electronic device (10) is integrally within said body (2) and/or within said collecting portion (3).

According to another non-limiting aspect, after the step of positioning (1002), of the step of manufacturing (1000) the body (2) and of the step of manufacturing (1001) the collecting portion (3), said body (2) and/or said collecting portion (3) defines a covering layer (4) of said at least one wireless identification electronic device (10), optionally said covering layer (4) extending around said at least one wireless identification electronic device (10) in a plurality of radial directions, extending in a direction oblique, preferably orthogonal, to the main development direction (X) of said body (2).

According to another non-limiting aspect, the step of manufacturing (1000) the body (2) and the step of manufacturing (1001) the collecting portion (3) determine a conjunction of the collecting portion (3) to said body (2) and/or determine an integral and/or seamless realization between the collecting portion (3) and the body (2).

According to another non-limiting aspect, the step of manufacturing (1001) the collecting portion (3) comprising:
a first sub-step of manufacturing a supporting portion (3a) for said swab (3b);
a second sub-step of manufacturing a swab (3b) preferably having one among the following properties: hydrophilic, hydrophobic or neutral, preferably comprising a plurality of fibers provided with hydrophilic capacity, and optionally being a step of flocking, and/or of fiber winding and/or of arrangement of a foam.

According to another non-limiting aspect, the step of positioning (1002) comprising a positioning of said wireless identification electronic device (10) within said supporting portion (3a).

According to another non-limiting aspect, said covering layer (4) is part of said supporting portion (3a).

According to another non-limiting aspect, said first sub-step of manufacturing the supporting portion (3a) occurs simultaneously with the step of manufacturing the body (2).

According to another non-limiting aspect, said at least one wireless identification electronic device (10) comprises a respective outer surface (10x).

According to another non-limiting aspect, said body (2) presents a respective outer surface (2x).

According to another non-limiting aspect, said step of positioning (1002) determines, following the completion at least of the step of manufacturing (1000) the body (2), the positioning of the outer surface (10x) of said at least one wireless identification electronic device (10) in substantial contact with the outer surface (2x) of the body (2) or the positioning of the outer surface (10x) of said at least one wireless identification electronic device (10) in such a way that at least part of said outer surface (10x) lies in correspondence of said outer surface (2x) of the body (2) and/or protrudes partially outside said body (2).

According to another non-limiting aspect, said step of positioning (1002) determines the positioning of said at least one wireless identification electronic device (10) in substantial correspondence of said ending portion (2e) or in correspondence of said gripping portion (2i).

According to another non-limiting aspect, the method comprises a step (1004) of realization of a weakening portion (5) on said body (2), the weakening portion being configured for facilitating a separation between at least part of the body (2) and the collecting portion (3).

According to another non-limiting aspect, the step of manufacturing (1000) the body (2) comprises a step of realization of a first half-body (7) and of a second half-body (8) different one another and selectively couplable, for defining a assembled configuration of the body (2), and selectively separable the one from the other, for defining a disassembled condition of the body (2), and wherein the step of manufacturing (1001) the collecting portion (3) comprises a realization and an engagement of the collecting portion (3) both on a first end of the first half-body (7) and on a first end of the second half-body (8).

According to another non-limiting aspect, the step of manufacturing (1001), of collection (3) comprises a realization and an engagement of the collecting portion (3) both on a first end of the first half-body (7) and on a first end of the second half-body (8) in such a way that said collecting portion (3) is selectively separable, after a separation of the first half-body (7) from the second half-body (8), for defining two half collecting portions respectively realized on the first half-body (7) and on the second half-body (8).

According to another non-limiting aspect, the assembly of the step of positioning (1002) and of the step of manufacturing (1000) the body (2) and/or of manufacturing (1001) the collecting portion (3), determines the arrangement of said at least one wireless identification electronic device (10) at least partially in said first half-body (7) or in correspondence of the collecting portion (3), preferably of the supporting portion (3a), joined to said first half-body (7) and/or realized integrally and/or seamlessly with said first half-body (7),
or determines the arrangement of the wireless identification electronic device (10) at least partially in said second half-body (7) or in correspondence of the collecting portion (3), preferably of the supporting portion (3a), joined to said first half-body (7) and/or realized integrally and/or seamlessly with said first half-body (7).

According to another non-limiting aspect, the assembly of the step of positioning (1002) and of the step of manufacturing (1000) the body (2) and/or of manufacturing (1001) the collecting portion (3), determines the arrangement of the first wireless identification electronic device (10) in correspondence of said first half-body (7) and of the second wireless identification electronic device (10) in correspondence of the second half-body (8), and/or
determines the arrangement of the first wireless identification electronic device (10) in correspondence of the half-collecting portion realized on the first half-body (7) and of the second wireless identification electronic device (10) in correspondence of the half-collecting portion realized on the second half-body (8), and/or
determines the arrangement of the first wireless identification electronic device (10) in correspondence of the first half-body (7) and of the second wireless identification electronic device (10) in correspondence of the half-collecting portion realized on the second half-body (8).

According to another non-limiting aspect, the step (1004) of realization of said weakening portion (5) determines the realization of said weakening portion (5) in a position axially translated, along said main development direction (X) of the body (2), with respect to the position assumed by said at least one wireless identification electronic device (10).

According to another non-limiting aspect, the step of positioning (1002) of said at least one wireless identification electronic device (10) is a step of positioning of a wireless identification electronic device (10) of substantially three-dimensional shape such that a main extension axis of said at least one wireless identification electronic device (10) results aligned to the main development direction (X) of the body (2).

According to another non-limiting aspect, said at least one wireless identification electronic device (10) is of passive type, or of active type, or of semi-active type or of semi-passive type; the step of positioning (1002) being specifically destined to allow a storage, in a memory of said at least one wireless identification electronic device (10), of tracking data of said sample and/or of the device (1) and/or of analysis method of said sample and/or of collection date of said sample, and/or of transport conditions of said sample.

According to another non-limiting aspect, said at least one wireless identification electronic device (10) is of a read only type, or is configured for operating in read only mode, in such a way that the data written in said memory can be only read, or wherein said at least one wireless identification electronic device (10) is of read-write type, or is configured for operating in read-write mode, in such a way that the data written in said memory can be read and written.

According to another non-limiting aspect, the method comprises a step (1003) of arrangement of at least one tag (12) on said device (1), said tag (12) having at least one alphanumeric and/or visual code; said alphanumeric and/or visual code representing at least part of the data stored in the memory of said at least one wireless identification electronic device (10) and/or being destined, when electronically read, to address a reader device (100) to the retrieval, in particular automated, of said data stored in the memory of said at least one wireless identification electronic device (10) or to the retrieval of an electronic copy of said data stored in the memory of said at least one wireless identification electronic device (10).

According to another non-limiting aspect, the step (1003) of arrangement of at least one tag (12) on said device (1) comprising a positioning of said tag (12) on the body (2) or on a gripping and/or closing element of a container (20) for said device (1), fixed to said body (2) in correspondence of said gripping portion (2i) or of a portion of said body (2) opposed with respect to said ending portion (2e).

According to another non-limiting aspect, the step of manufacturing (1000) the elongated body (2) and/or the step of manufacturing (1001) said collecting portion (3), preferably of said supporting portion (3a), comprises manufacturing said elongated body (2) and/or said collecting portion (3), preferably said supporting portion (3a), partially or integrally, with at least one among the materials of the following list: polyester and derivatives, for example polybutylene terephthalate (PBT) or polyethylene terephthalate (PET); polyether ether ketone (PEEK); polyphenylenesulfide (PPS); acrylonitrile butadiene styrene (ABS); polyurethane (PU); polyvinylchloride (PVC); polymethylmethacrylate (PMMA); high or low density polyester (HDPE, LDPE); polycarbonate; polystyrene; polyamide, in particular aliphatic polyamide, for example Nylon.

According to another non-limiting aspect, the step of manufacturing (1000) the elongated body (2) and/or the step of manufacturing (1001) said collecting portion (3), preferably of said supporting portion (3a), comprises manufacturing said elongated body (2) and/or said collecting portion (3), preferably said supporting portion (3a), partially or integrally, with a thermoplastic or thermosetting material.

According to the present disclosure is described a container (20) configured for a device (1) according to one or more of the aspects herein described.

According to another non-limiting aspect, said container (20) presents a substantially axial extension.

According to another non-limiting aspect, said extension substantially axial is equal to or greater than the extension of said elongated body (2) along said main development direction (X) and/or equal to or greater than the sum of the extension of said elongated body (2) along said main development direction (X) with the extension of said collecting portion (3).

According to another non-limiting aspect, said container (20) is configured for being closed, preferably hermetically, by a stopper (16).

According to another non-limiting aspect, said stopper (16) is connected to, preferably unmovably connected to, said device (1).

According to another non-limiting aspect, said container (20) presents a lateral wall defining a cavity in use housing said device (1) and configured for allowing the transmission and reception of a wireless electromagnetic signal towards the device (1) and from the device (1) when contained in said cavity.

According to the present disclosure is also described a method of electronic reading of electronic data written in wireless identification electronic device (10) contained in a device (1) for the collection and the transfer of samples according to one or more of the aspects herein described, the method comprising:
a step of positioning of the wireless identification electronic device (10) in proximity of a reader device (100);
a step of transmission of a predefined query signal from the reader device (100) towards the wireless identification electronic device (10);
a step of reception of the query signal, the reception occurring on said at least one wireless identification electronic device (10), the step of reception determining the electronic access to a memory of said at least one wireless identification electronic device (10) and the loading of at least part of the data contained in said memory, for a successive retransmission of a reply signal;
a step of transmission of the reply signal towards the reader device (100), said reply signal containing at least part of the data stored in said memory.

According to another non-limiting aspect, said loading is a loading electronically carried out in an automatic way by a control unit of said at least one wireless identification electronic device (10).

According to another non-limiting aspect, the access to the memory is automatically carried out upon reception of the query signal.

According to another non-limiting aspect, the step of transmission occurs in an automatic way after the electronic access to the memory.

According to another non-limiting aspect, the method comprises:
a step of positioning of a first wireless identification electronic device (10) in proximity of a reader device (100);
a step of positioning of a second wireless identification electronic device (10) in proximity of said reader device (100);
a step of transmission of a predefined query signal from the reader device (100) towards the first and towards the second wireless identification electronic device (10);
a step of reception of the query signal, the reception occurring on the first and on the second wireless identification electronic device (10), the step of reception determining the electronic access to a memory of the first and of the second wireless identification electronic device (10) and the loading of at least part of the data contained in said memory, for a successive retransmission of a first, respectively a second, reply signal;
a step of transmission of a first reply signal from the first wireless identification electronic device (10) towards the reader device (100), said first reply signal containing at least part of the data stored in said memory of the first wireless identification electronic device (10);
a step of transmission of a second reply signal from the second wireless identification device (10) towards said reader device (100), said second reply signal containing at least part of the data stored in said memory of the second wireless identification electronic device (10);
a step of electronic comparison of the second reply signal to said first reply signal.

According to another non-limiting aspect:
if at least part of said second reply signal is identical to a respective at least part of said first reply signal, it is determined a consistency of coupling between said first and said second wireless identification device (10), or
if no part of said second reply signal is identical to a respective part of said first reply signal, it is determined an absence of consistency of coupling between said first and said second wireless identification device (10).

According to the present disclosure is also described a method of electronic writing of electronic data in at least one wireless identification electronic device (10) of a device (1) for the collection and the transfer of samples according to one or more of the aspects herein described, the method comprising:
a step of writing of electronic data within the memory of said at least one wireless identification electronic device (10);
optionally, a step of configuration of said at least one wireless identification electronic device (10) in such a way that this electronic device is arranged in read only mode, so that the electronic data written in the memory can no longer be altered.

According to another non-limiting aspect, the step of writing of said data occurs before said at least one wireless identification electronic device (10) is introduced in the elongated body (2) and/or in the collecting portion (3).

According to another non-limiting aspect, the step of writing of said data occurs after said at least one wireless identification electronic device (10) has been introduced in the elongated body (2) and/or in the collecting portion (3), preferably after the elongated body (2) and/or the collecting portion (3) have been moulded, in particular co-moulded and/or injection moulded and/or overmoulded and/or moulded by insertion, in a mould wherein said at least one wireless identification electronic device (10) is included.

According to the present disclosure is also described an equipment for the manufacturing of a device (1) for the collection and the transfer of samples of biological, microbiological or chemical material, preferably for the manufacturing of a device (1) for the collection and the transfer of samples of biological, microbiological or chemical material according to one or more of the aspects herein described.

According to another non-limiting aspect, said equipment is configured and specifically destined to cause the execution of the steps of the method according to one or more of the aspects herein described.

According to the present disclosure is described also a control unit, configured for causing the activation of an equipment for the manufacturing of a device (1) for the collection and the transfer of samples of biological, microbiological or chemical material according to one or more of the method aspects herein described.

According to the present disclosure is also described a software program, comprising portions of software code that when executed are destined to cause the execution of the method according to one or more of the aspects herein described.

### FIGURES

The object of the present disclosure will be described in the detailed description, which refers to some preferred embodiments of device 1. In particular, these preferred embodiments are described with reference to the attached figures; a brief description of the figures is provided hereinafter.

FIG. 1 shows a lateral view of a device object of the present disclosure, in a first embodiment.

FIG. 2 shows a sectional view of the device of FIG. 1, along the lines II-II.

FIG. 3 shows a sectional view of another embodiment of the device object of the present disclosure.

FIG. 4 shows a lateral view of another embodiment of the device object of the present disclosure.

FIG. 5 shows a sectional view of the device of FIG. 4, along the lines V-V.

FIG. 6 shows a lateral view of another embodiment of the device object of the present disclosure.

FIG. 7 shows a lateral view of a part of a particular embodiment of a device object of the present disclosure, wherein is present a weakening portion.

FIG. 8 shows a lateral view of an embodiment of the device object of the present disclosure included in a container.

FIG. 9 shows a lateral view of another embodiment of the device object of the present disclosure, wherein a first and a second wireless identification electronic device are present.

FIG. 10 shows a lateral view of a part of a particular embodiment of a device object of the present disclosure, wherein is present a weakening portion, and wherein are present a first and a second wireless identification electronic device.

FIG. 11 shows a lateral view of another embodiment of the device object of the present disclosure included in a container.

FIG. 12 shows a sectional view of the device of FIG. 11.

FIG. 13 shows a section lateral view of a detail of a particular embodiment of the device object of the present disclosure.

FIG. 14 shows a section lateral view of a detail of a particular embodiment of the device object of the present disclosure, wherein are present a first and a second wireless identification electronic device respectively subjected to a process of moulding in the first half-body and in the second half-body opposed to the first.

FIG. 15 shows a short flow chart of a manufacturing process of a device object of the present disclosure.

FIG. 16 shows a schematic representation of a mould and of a relative device object of the present disclosure manufactured by moulding.

FIG. 17 shows a schematic representation of a reader device for wireless identification electronic devices (RFID tags) configured for executing electronic comparisons of data.

FIG. 18 shows a schematic representation of a non-limiting embodiment of a reader device.

FIGS. 19, 20, 21, 22, show specific embodiments of the device object of the present disclosure, wherein are present a first and a second wireless identification electronic device.

### DETAILED DESCRIPTION

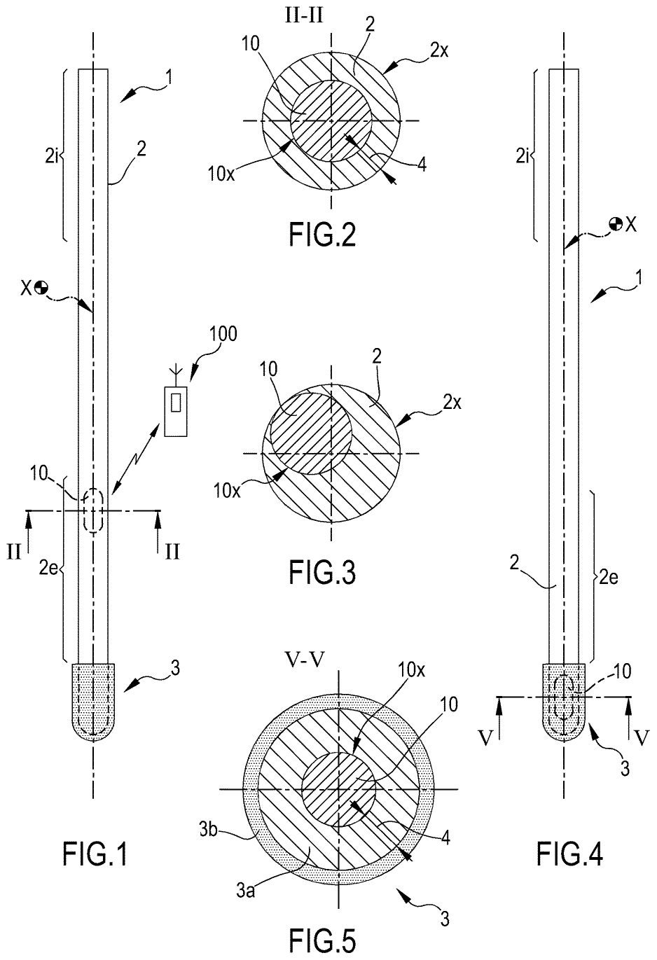

With reference to FIG. 1, with reference number 1 is indicated as a whole a device 1 for the collection and the transfer of samples, preferably but non-limiting thereto of biological, microbiological or chemical material.

For the purposes of the present disclosure, for “biological material” it is intended a material, for example wet material, in particular and not limiting thereto fluid and/or liquid, containing at least one among a secretion of a tissue, a tissue sample, tissue cells, blood, etc. . . . , of a human or animal being.

In a preferred embodiment, the device 1 is mainly destined to allow the collection of biological samples from a nasal cavity, in particular from a portion of mucosa of the nasal cavity and/or nasopharynx.

The device 1 herein described can be advantageously used, for example, for the execution of screening and/or test of presence of coronavirus, in particular, and non-limiting thereto, of virus of SARS-COV2 type (base variant and its subsequent variants). The device 1 herein described realizes therefore a so-called nasopharyngeal swab.

The device 1 herein described can also be used for activities of collection of nonbiological substances or compounds, for example inorganic substances, from surfaces such as for example roads, walls, vehicles, etc.

The Applicant has noted that for keeping a correct cataloging of the sample collected through the device 1 is imperative that there is consistency of association with the device 1 itself, rather than with the container thereof.

The Applicant has also noted that the use of wireless identification electronic devices, commonly known as “RFID tags” in association with device 1 for the collection and the transfer of samples allows to optimize such association by significantly reducing the risk of wrong association between data of the collected sample and device 1.

RFID tags are examples of wireless identification electronic devices (in the present description identified by the numerical reference 10), configured for receiving and/or transmitting and/or storing electronic data on a wireless communication channel, towards an electronic device which constitutes a transmitter of electronic data towards at least one wireless identification electronic device and/or which constitutes a receiver of electronic data transmitted by the at least one wireless identification electronic device.

In detail, the device 1 object of the present disclosure, in a general embodiment thereof, is provided at least with:
a body 2, preferably of elongated type that therefore, preferably, extends at least partially along a main development direction X substantially axial and comprising at least one gripping portion 2i destined to be grasped by a user and at least one ending portion 2e;
a collecting portion 3, arranged in correspondence of said ending portion 2e and configured for collecting and/or absorbing and/or retaining and/or releasing a quantity of a sample, preferably a sample of biological, microbiological or chemical material.

In the following portion of the description, reference will be made mainly to body 2 in elongated form as the preferred embodiment of the above-mentioned body. It should be intended that where possible, the characteristics of the body 2 in elongated form also extend to bodies 2 also in non-elongated form.

The elongated body 2 is preferably realized in plastic material; this technical feature allows to have an elongated body 2 extremely economical to manufacture. The elongated body 2 is substantially flexible, in order to be able to adapt at least partially to a bend of the nasal cavity, of the naris and/or of the nasopharynx.

The flexibility of the elongated body 2 is mainly given along a direction substantially parallel to the axis X. This means that such a body can be flexed to describe a curve that departs from said axis X. Along a direction substantially orthogonal with respect to the axis X, preferably the body 2 in both elongated and non-elongated form is nearly incompressible.

For example, the elongated body 2 may have a substantially circular transversal section, and develop along the axis X with a substantially constant diameter, for example and not limited thereto comprised between 1.5 mm and 3 mm, more preferably between 2 mm and 2.5 mm. When not of circular transversal section, the shape of the transversal section may be enclosed in a circumference whose diameter respects the above-described limits.

If the body is not of elongated type, in particular an extension along an axis orthogonal to the axis X can be substantially equal to the extension of the body along said axis X.

The elongated body 2 is represented in FIG. 1 as having substantially uniform thickness. This should not be intended in a limiting way, since in an alternative embodiment, the elongated body 2 can have a diameter, or more generally a transversal section, of area that is not constant as you move along the axis X. When realized in plastic material, the elongated body 2 can be realized by moulding, in particular by injection moulding or co-moulding or even by overmoulding, in particular by insertion or with core loss (lost core).

For the purposes of the present disclosure, for overmoulding is intended a process wherein, on a substrate, for example of plastic material which is in turn molded, occurs a process of moulding of a covering layer directly on the substrate so that it is possible to realize a finished product.

The overmoulding comprises the moulding by insertion and the moulding by core loss.

For the purposes of the present disclosure, for moulding by insertion is intended a process wherein a part realizing the substrate, for example metallic, is first placed in a mould and then in said mould the process of moulding occurs to create a finished product. The substrate remains visible.

In the moulding by core loss, on the other hand, the substrate material is dissolved or melted into the coating material.

In an embodiment, the elongated body 2 can be realized by a 3D moulding process.

The 3D moulding process can be conveniently carried out by using a polymeric plastic material for example and not limiting thereto of the polyester type, for example PBT, or PEEK, or PPS, or polycarbonate, or ABS, or PVC, or PMMA.

Non-limiting examples of plastic materials that can be used for the partial or full manufacturing of the elongated body 2, include:
polyester and derivatives, for example polybutylene terephthalate (PBT) or polyethylene terephthalate (PET);
polyether ether ketone (PEEK);
polyphenylenesulfide (PPS);
acrylonitrile butadiene styrene (ABS);
polyurethane (PU);
polyvinylchloride (PVC);
polymethylmethacrylate (PMMA);
high- or low-density polyester (HDPE, LDPE);
polycarbonate;
polystyrene;
polyamide, in particular aliphatic polyamide, for example Nylon.

It is therefore clear that plastic materials that can be used for the realization of the elongated body 2 can be of both thermoplastic type and thermosetting type (for example, PU).

It should be intended that where in the present disclosure generic reference is made to a plastic material, the specific typologies of plastic material herein described in relation to 3D moulding of the elongated body may be equivalently used, alternatively or in addition to other specifically described material typologies.

Superficially, the elongated body 2, in particular in correspondence of the gripping portion 2i, can be provided with elements (for example knurls) designed to increase the grip with the fingers and/or palm of the user's hand. In an embodiment, at least part of the gripping portion 2i can be provided with a rubbered layer.

The collecting portion 3 comprises preferably:
a swab 3b, that in particular has preferably one among the following substantial properties: hydrophilic, hydrophobic or neutral;
a supporting portion 3a for the swab 3b.

In a preferred embodiment, the swab 3b comprises a plurality of fibers provided with hydrophilic capacity, and optionally being a flocked swab 3b, and/or with wound fiber, and/or in foam.

A swab 3b with neutral property shows absence of pronounced hydrophilicity and absence of pronounced hydrophobic properties.

In a preferred configuration, if it is observed the device 1 along the direction X, the supporting portion 3a is radially internal with respect to the swab 3b. The latter surrounds the supporting portion 3a.

The supporting portion 3a can be intended as a seamless continuation of the elongated body 2.

Materials that can be used for the partial or full manufacturing, of the collecting portion 3, and in particular for the partial or full realization, of the supporting portion 3a, are the ones above listed for the partial or full manufacturing, of the elongated body 2.

In an embodiment, the collecting portion 3 is configured and specifically destined to allow the collection of at least 40 ml, or 50 ml, or 70 ml, or 100 ml of fluid. This is in particular valid if the collecting portion 3 can be in form of sub-container. If the collecting portion 3 is of swab type, in particular flocked, typical quantities of samples, preferably fluids, that are collected can arrive to 50 μl or 70 μl, or 100 μl or up to 150 μl. This ensures a collection of fluid sufficient for reliably performing further tests, in particular diagnostic type tests and more in particular tests of nucleic acid amplification (NAAT) with a reverse reaction process of the polymerase chain. This, for example, makes suitable the collecting portion 3 for the collection of samples to carry out screening and/or tests for the presence of coronavirus and/or for forensic applications.

In a specific embodiment, the collecting portion 3, and in particular the swab 3b, can be pre-imbibed or impregnated with a substance, in particular with a reagent.

In case of a flocked swab, the flocking process is such that it allows the deposit of fibers in an electrostatic field, which allows to deposit the fibers in an ordered manner, perpendicularly to the surface of the supporting portion 3a.

In an embodiment, fibers are selected from a list of materials comprising synthetic and natural materials, and in particular from a list of materials comprising at least one among: rayon, polyester, polyamide, carbon fiber, alginate, cotton, silk or mixtures of them, sponge.

In the device 1 object of the present disclosure, at least one between the elongated body 2 and/or the collecting portion 3 is realized by moulding, preferably injection moulded and/or overmoulded, in particular moulded by insertion or with core loss (lost core).

As already mentioned, the device 1 herein described comprises at least one wireless identification electronic device 10 (RFID tags), configured for receiving and/or transmitting and/or storing electronic data on a wireless communication channel. This wireless identification electronic device 10 is arranged, at least partially, within said elongated body 2 and/or within said collecting portion 3.

The use of the clause “and/or” in the above sentence is justified in that the Applicant has conceived different embodiments of the device 1 that provide the positioning of the wireless identification electronic device 10 in correspondence of the elongated body 2, or in correspondence of the collecting portion 3, or partially in correspondence of the elongated body 2 and partially in correspondence of the collecting portion 3.

There may be more wireless identification electronic devices 10 as shown below:
a first wireless identification electronic device 10 and at least one second wireless identification electronic device 10 arranged at least partially within said first body 2; or
a first wireless identification electronic device 10 arranged at least partially within said first body 2 and at least one second wireless identification electronic device 10 arranged within said collecting portion 3;
a first wireless identification electronic device 10 and at least one second wireless identification electronic device 10 arranged within said collecting portion 3;
a first wireless identification electronic device 10 arranged within said collecting portion 3 and at least one second wireless identification electronic device 10 arranged at least partially within said first body 2.

The following description shows some preferred and non-limiting embodiments of the device 1 making specific reference to the attached figures.

If applicable, what is described below applies to the at least one and preferably all the wireless identification electronic devices 10 present on the device 1. Therefore, no specific difference in description will be made between the embodiments wherein is present a single wireless identification electronic device with respect to the ones wherein are present more wireless identification electronic devices, in order not to excessively increase the length of the present text.

In particular in FIG. 1 is represented a lateral view of a first embodiment of a device 1 wherein the wireless identification electronic device 10 is arranged inside the elongated body 2. FIG. 2, which shows a sectional view along the lines II-II of FIG. 1 shows that the wireless identification electronic device 10 is centered on the axis X along which extends the elongated body 2. In the specific embodiment of FIG. 1 the wireless identification electronic device 10 assumes a substantially three-dimensional shape, with a body having a main extension direction parallel to the main extension direction X of the elongated body 2. At least part of the transversal section (plane orthogonal to the direction X) of the wireless identification electronic device 10 assumes substantially circular shape. This means that at least part of the wireless identification electronic device 10 is cylindrical. In FIG. 2 there are an outer surface 2x of the elongated body 2 and an outer surface 10x of the wireless identification electronic device 10.

In an embodiment of FIGS. 1 and 2, the wireless identification electronic device 10 is positioned, or is enclosed, integrally within the elongated body 2, and in particular is arranged in the ending portion 2e of the above-mentioned body.

The elongated body 2 defines a covering layer 4 of the wireless identification electronic device 10; consequently, the outer surface 10x of the wireless identification electronic device 10 is inside the elongated body 2, is surrounded by the covering layer 4, and is thus in a position radially internal with respect to the outer surface 10x.

The thickness of the covering layer 4 in FIG. 2 is represented as uniform; this results from a particular and non-limiting combination of the shape of the transversal sections of the wireless identification electronic device 10 and of the elongated body 2, that in particular in FIG. 2 assumes an at least partially substantially cylindrical shape. This form should not be intended in a limiting way.

It can therefore be asserted that in some embodiments of the device 1, the covering layer 4 extends around the wireless identification electronic device 10 in a plurality of radial directions, extending in a direction oblique, preferably orthogonal, to the main development direction X of the elongated body 2.

FIG. 3 shows a sectional view along the axis X of an alternative embodiment of the device 1. In FIG. 3, while remaining the wireless identification electronic device 10 in correspondence of the elongated body, it is noted that such wireless identification electronic device 10 lies not axially centered on the axis X of the elongated body 2. In particular the non-limiting embodiment represented in FIG. 3 is such that part of the outer surface 10x of the wireless identification electronic device 10 is in substantial contact with the outer surface 2x of the elongated body 2. This means that moreover the outer surface 10x of the wireless identification electronic device 10 can be observable by eye, observing laterally the device 1 from the right angle.

In further embodiments, not represented in the attached figures, the outer surface 10x of the wireless identification electronic device 10 protrudes partially outside said elongated body 2.

FIG. 4 shows a lateral view of another embodiment of a device 1, wherein the wireless identification electronic device 10 is arranged integrally inside the collecting portion 3.

In this case it is the collecting portion 3, and in particular the supporting portion 3a of the collecting portion 3, that defines the covering layer 4 of the wireless identification electronic device 10. Consequently, the outer surface 10x of the wireless identification electronic device 10 is inside the collecting portion 3 and in particular of the supporting portion 3a. The wireless identification electronic device 10 lies in a position radially internal with respect to the outer surface 10x.

As shown in detail in FIG. 5, which represents a section of the device 1 according to FIG. 4 along the lines V-V, the thickness of the covering layer 4 can be uniform; this results from a particular and non-limiting combination of form of the transversal sections of the wireless identification electronic device 10 and of the collecting portion 3, in particular of the supporting portion 3a of the collecting portion.

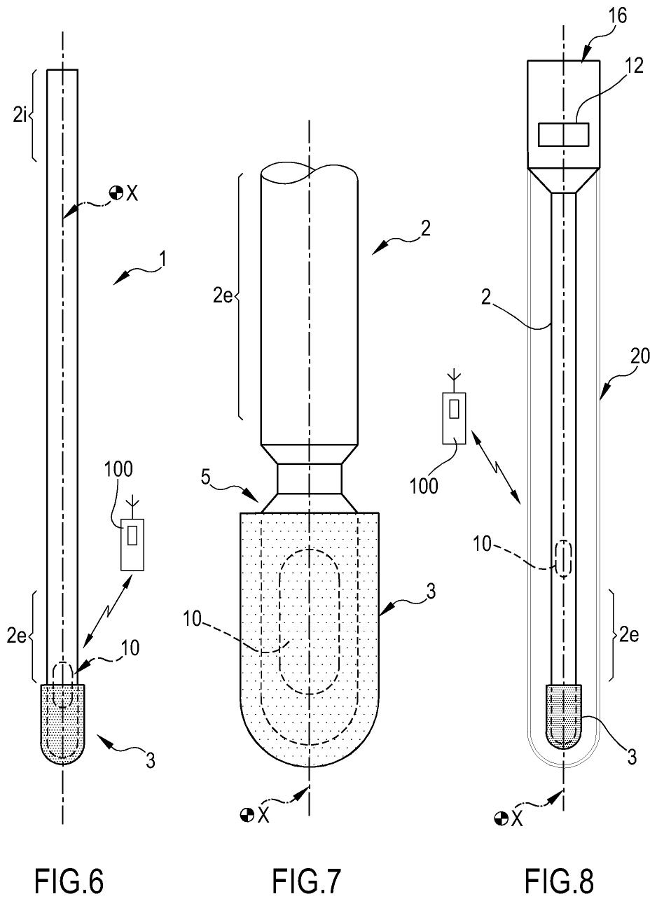

FIG. 6 shows a further embodiment of the device 1 object of the present disclosure. In this embodiment the wireless identification electronic device 10 is arranged between the elongated body 2 and the collecting portion 3: part of the wireless identification electronic device 10 is in the elongated body 2, in particular in correspondence of the ending portion thereof; part of the wireless identification electronic device 10 is in the collecting portion 3, for example being, integrally contained in the supporting portion 3a of the collecting portion 3 or positioned in such a way that part of the outer surface 10x of the wireless identification electronic device 10 is in correspondence of the interface surface between the supporting portion 3a and the swab 3b. Consequently, also in this embodiment the wireless identification electronic device 10 can be axially aligned on the main development direction X of the elongated body 2, or—alternatively—be axially misaligned with respect to this direction.

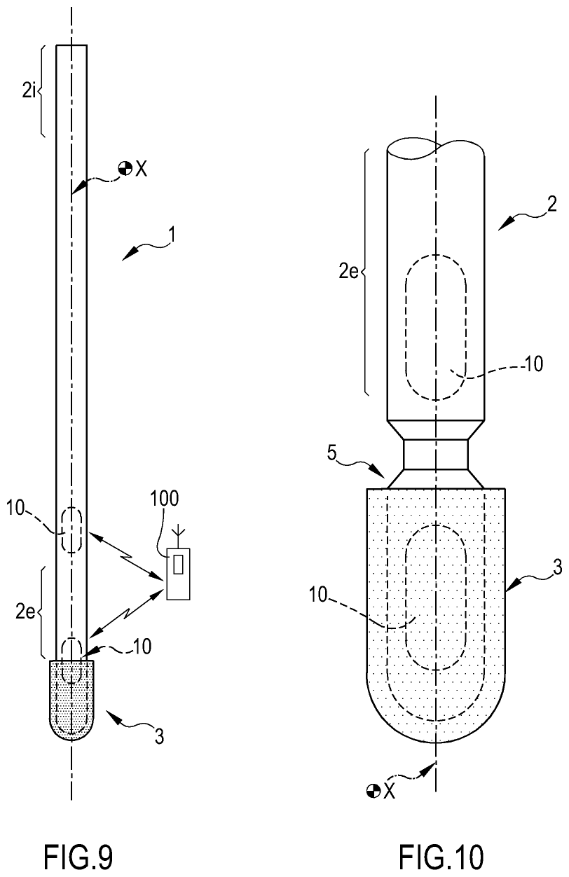

In FIG. 9 is shown a particular embodiment of the device 1 object of the present disclosure wherein:
a first wireless identification electronic device 10 is positioned in correspondence of an area between the ending portion 2e of the body 2 and the collecting portion 3, and
a second wireless identification electronic device 10 is positioned in correspondence of an intermediate area between the gripping portion 2i and the ending portion 2e.

FIG. 7 shows a particular embodiment of a device 1 wherein the elongated body 2 is provided with a weakening portion 5.

The purpose of the weakening portion 5 is to facilitate the separation of the collecting portion 3 from at least part of the elongated body 2 by fracture.

In an embodiment of FIG. 7, the weakening portion 5 is arranged in correspondence of the ending portion 2e of the elongated body 2; this configuration shall not be intended in a limiting way: in fact it is possible that the weakening portion 5 is arranged in correspondence of portions of the elongated body 2 more backward with respect to the ending portion 2e, for example in correspondence of a substantially central area of the elongated body 2.

The weakening portion 5 can be realized with various techniques one of which, represented in FIG. 7, comprises a local reduction of the area of the transversal section of the elongated body 2. For example, if the elongated body 2 is of circular section (assuming for simplicity that the diameter of the elongated body 2 is constant along its entire development), the weakening portion 5 may advantageously be realized by a local diameter reduction with respect to the diameter of the remaining part of the elongated body. It appears clear that the reduction of the diameter, causes a greater local fragility that facilitates the separation of the elongated body 2 from the collecting portion 3.

In FIG. 7 is represented a specific and non-limiting embodiment of the device 1 wherein, the elongated body 2 is provided with the weakening portion and wherein the wireless identification electronic device 10 is positioned in correspondence of the collecting portion 3.

It shall not be intended in a limiting way that the weakening portion 5 is associated to a wireless identification electronic device 10 positioned in correspondence of the collecting portion 3; in fact, the elongated body 2 could be provided with a weakening portion 5, even having the wireless identification electronic device 10 arranged in correspondence of the elongated body 2 or partially in correspondence of the elongated body 2 and partially in correspondence of the collecting portion 3, in the above-described modes.

If are present at least two wireless identification electronic devices 10, these can be positioned upwards and downwards of the weakening portion 5, in such a way that also if the collecting portion 3 should be separated by fracture from the remaining portion of the body 2, a unique electronic track can be kept of which body 2 has housed the sample contained in a specific collecting portion 3. This solution is represented in FIG. 10. Herein is clearly represented a first wireless identification electronic device 10 arranged in correspondence of the collecting portion 3, then downwards of the weakening portion, and a second wireless identification electronic device 10 arranged in correspondence of the ending portion 2e of the body 2.

The wireless identification electronic device 10 comprises first of all a memory that is configured for storing electronic data, an antenna and a physical support, typically realized in plastic material, that integrates the memory and, if it is the case, the antenna.

Preferably the memory is a non-volatile type memory, for example and non-limiting thereto a EEPROM or FRAM memory.

In an embodiment, the wireless identification electronic device 10 can be of active, passive, semi-active or semi-passive type.

A wireless identification electronic device 10 of passive type is configured for replying with a transmission of a determined wireless signal only when queried by a reader that transmits a predefined query signal. In this case, the power of the signal received by the wireless identification electronic device 10 is sufficient to generate enough power to transmit a reply signal. Such a passive device is without a battery.

A wireless identification electronic device 10 is also able to be written by a transmitter device; in this case a wireless signal is transmitted toward the wireless identification electronic device 10 and the data carried via the above-mentioned signal are stored in the memory of the device.

A wireless identification electronic device 10 of active type is typically provided with an internal powering battery, and further than the memory typical also of the wireless identification electronic device 10, has optionally more antennas for receiving and/or transmitting and/or storing signals on multiple wireless channels. The range of the transmission of the wireless identification electronic device 10 of active type, is greater with respect to the range of the transmission of the wireless identification electronic device 10 of passive type.

A wireless identification electronic device 10 of semi-passive type is provided too with a memory and a battery, but the battery is used only to power a microchip, if present, or auxiliary apparatuses; the battery is not used to power the transmission of the signal provided as a reply to a query signal. In other words, upon query, a wireless identification electronic device 10 of semi-passive type behaves in a way in principle identical to a wireless identification electronic device 10 of passive type.

A wireless identification electronic device 10 of semi-active type is provided too with a memory and a battery, but in this case the battery is destined also to supply power for the transmission of the reply signal to the query signal. Usually, the wireless identification electronic device 10 is deactivated and is powered only upon reception of a predefined transmission signal, operating in a way similar to a wireless identification electronic device 10 of passive type.

Preferably, but non-limiting thereto, the Applicant prefers to use wireless identification electronic devices 10 of passive type, that advantageously allow to reduce the costs associated with the manufacturing of the device 1 and that they can be more miniaturized with respect to others.

The wireless identification electronic device 10 is capable of operating, in principle, on a wide variety of frequency ranges—from LF to frequencies in the microwave range.

It is herein below proposed a non-limiting example of frequency bands that can be used for wireless identification electronic devices 10:
125/134 kHz (LF)
13,56 MHz (HF)
433 MHz (UHF, typically used for active tags)
860-960 MHz (UHF)
2,4 GHz (UHF, typically used for active or passive tags)
5,8 GHz (SHF, generally used only for active tags)

The Applicant notes that the wireless identification electronic devices 10 that operate in LF (125/134 kHz) and HF (13,56 MHz) bands are provided for in the ISO standards as passive.

In an embodiment, the wireless identification electronic device 10 can be, or can be made to operate, in read only mode. The read only mode is a mode in which the data contained in the memory of the wireless identification electronic device 10 can only be read and not written.

In an alternative embodiment, the wireless identification electronic device 10 can be, or can be made to operate, in read/write mode. The read/write mode is such that at least part of the data contained in the memory of the wireless identification electronic device 10 can be both written and read.

In a particular embodiment, the wireless identification electronic device 10 can comprise a memory wherein:
a first part of the memory can be written and/or read;
a second part of the memory can be only read.

In an embodiment, the data that can be stored in the memory are for example tracking data of said sample and/or of the device 1 and/or of analysis method of said sample and/or of collection date of said sample, and/or of transport conditions of said sample.

The data that can be stored in the memory can also include a univocal identification code of the wireless identification electronic device 10. This code can be attributed by the producer of the wireless identification electronic device 10 or by the producer of the device 1 herein described. This code is stored so that it cannot be altered; in an embodiment wherein are present the first part of the memory and the second part of the memory, this univocal identification code is stored in the second part of the memory.

The presence of a univocal identification code allows to safely track the device herein described; the possibilities of alteration, especially unintentional, of the univocal identification code only electronically readable are almost negligible. Also, the fact that the univocal identification code is stored in an only readable area of a memory of a wireless identification electronic device 10 integrated into a body 2 and thus not easily removable, it also reduces the risk of voluntary alteration thereof-for example by replacement of the wireless identification electronic device 10.

The wireless identification electronic device 10 can be configured for transmitting a wireless electronic signal that includes or not said univocal identification code. In particular, there can be the following configurations:
at each query the wireless identification electronic device 10 transmits only the univocal identification code in the reply signal;
at each query the wireless identification electronic device 10 transmits in the reply signal at least part of the data of the memory, preferably of the first part of the memory, that do not include said univocal identification code;
according to the query signal, the wireless identification electronic device 10 transmits:
   a) only the univocal identification code; or
   b) transmits at least part of the data of the memory, preferably of the first part of the memory, that do not include said univocal identification code; or
   c) transmits at least part of the data of the memory, preferably of the first part of the memory, that do not include said univocal identification code and the univocal identification code.

In the case referred to at item c), the transmission of the univocal identification code can:
follow, temporally, the transmission of at least part of the data of the memory, preferably of the first part of the memory, that do not include said univocal identification code and the univocal identification code, or alternatively
precede, temporally, the transmission of at least part of the data of the memory, preferably of the first part of the memory, that do not include said univocal identification code.

It appears then clear that the present disclosure also concerns a method of reading of electronic data written in the wireless identification electronic device 10 of a device 1 according to the present disclosure.

The method comprises first of all a step of positioning of the device 1 in proximity of a reader device 100. The distance required to allow the correct reception of the query signal by the wireless identification electronic device 10 depends at least on one of the following factors:
the technology with which is realized the wireless identification electronic device 10, in particular as a function of whether the latter is of active, passive, semi-active, semi-passive type;
the antenna gain of the reader device 100;
the shield factor provided by the possible presence of the container 20.

The shield factor shall be as limited as possible and for this reason the lateral wall of the container 20 is realized in a material specifically configured for determining a low absorption of electromagnetic signals transmitted by the reader device 100 towards the device 1 when placed within the cavity of the container 20 or received by the reader device 100 if transmitted by the device 1 placed within the cavity of the container 20.

Typically, for a wireless identification electronic device 10 of passive type can be reasonably estimated a distance of a few centimeters, and up to 30 cm, or up to 20 cm or up to 10 cm, as distance between the wireless identification electronic device 10 and the reader device 100 in order to allow a correct reception of the reply signal.

The method then provides a step of transmission of a predefined query signal from the reader device 100 towards the device 1; this step provides, if it is the case, the crossing—by the query signal—of the wall of the container 20 and/or of part of the elongated body 2 or of the collecting portion 3.

The method then provides a step of reception of the query signal, the reception occurring on the wireless identification electronic device 10; the step of reception determines the access to the memory of the wireless identification electronic device 10 and the retrieval of the data that shall be transmitted back with the reply signal. The access to the memory is automatically carried out upon reception of the query signal.

The method then provides a step of transmission of a reply signal towards the reader device 100; this step of transmission occurs in an automatic way after the access to the memory. This reply signal is a reply signal clearly transmitted on a wireless channel.

If the device 1 comprises a plurality of wireless identification electronic devices 10, may exist:
a step of positioning of a first wireless identification electronic device 10 in proximity of a reader device 100, and
a step of positioning of a second wireless identification electronic device 10 in proximity of said reader device 100.

The two above-described steps are substantially simultaneous since both the first and the second wireless identification electronic device 10 are in relative proximity due to the modest size of the device 1.

There is then a step of transmission of a predefined query signal by the reader device 100 towards the first and towards the second wireless identification electronic device 10; preferably, the query signal is a, and comes thus in substantial temporal simultaneity both on the first that on the second wireless identification electronic device 10. The difference of distance (first wireless identification electronic device 10—reader device 100) and (second wireless identification electronic device 10—reader device 100) produces an absolutely negligible delay of propagation for the purposes of the present disclosure.

Thus, there is a step of reception of the query signal, the reception occurring on the first and on the second wireless identification electronic device 10.

The step of reception determines the electronic access to a memory of the first and of the second wireless identification electronic device 10 and the loading of at least part of the data contained in said memory, for a successive retransmission of a first and respectively a second reply signal.

Thereafter, a step of transmission of a first reply signal by the first wireless identification electronic device 10 occurs towards the reader device 100.

The first reply signal contains at least part of the data stored in said memory of the first wireless identification electronic device 10, preferably the data not being part of the univocal identification code of the first wireless identification electronic device 10.

Also, a step of transmission of a second reply signal by the second wireless identification device 10 occurs towards said reader device 100.

The second reply signal contains at least part of the data stored in said memory of the second wireless identification electronic device 10, preferably the data not being part of the univocal identification code of the second wireless identification electronic device 10.

There can be then a step of electronic comparison of the second reply signal to said first reply signal, that is aimed at determining the consistency of coupling between the first and the second wireless identification electronic device 10 in terms of data coherence (i.e., if the first and the second half-body 7, 8 were part of a same body 2 or if a first and a second portion of the device 1 originally separated from the weakening portion 5 were part of a same device 1).

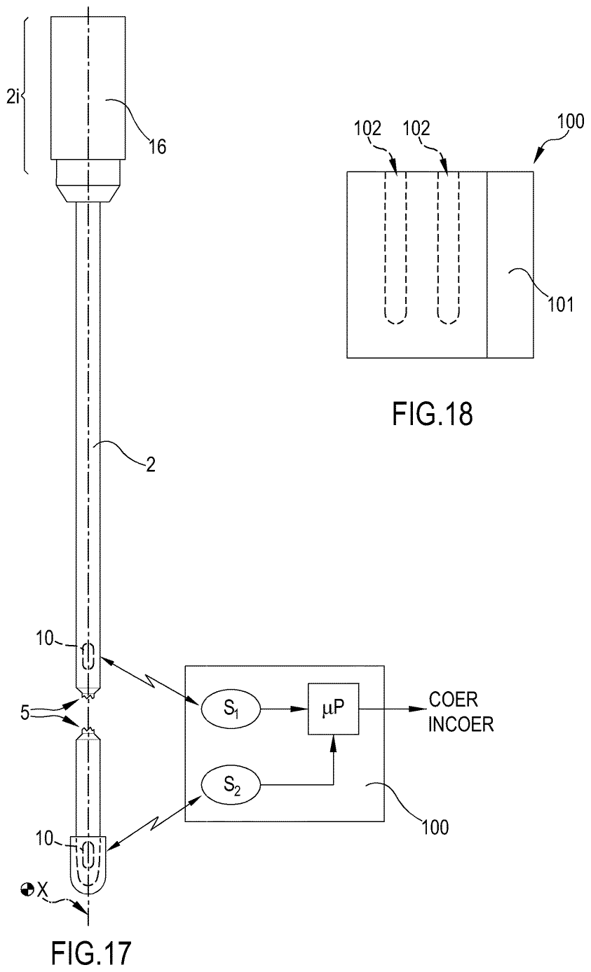

This step is schematically summarized, from the structural point of view by FIG. 17. Here is indicated a device 1 wherein is present a weakening portion 5 that has physically separated a first and a second wireless identification electronic device 10 respectively integrated upwards and downwards of the above-mentioned weakening portion 5. It is noted that in particular the collecting portion 3 contained on the bottom of a container 20. The first and the second reply signal are indicated in FIG. 17 with the reference S1 and S2. The control unit carries out the reading and the electronic comparison of the data contained in the first and in the second reply signal. If necessary, it carries out a decryption of the above.

If at least part (in particular, the part that excludes the univocal identification code) of said second reply signal is identical to a respective at least part of said first reply signal, it is determined a consistency of coupling between said first and said second wireless identification device 10.

Otherwise, if no part of said second reply signal is identical to a respective part of said first reply signal, it is determined an absence of consistency of coupling between said first and said second wireless identification device 10.

Should, in particular by configuration or constructive limits of the wireless identification electronic device 10, the step of transmission of the reply signal occur simultaneously with at least part of the step of reception of the query signal, there will be a wireless identification electronic device 10 of type or with full duplex behaviour, capable of operating in a simultaneous bidirectional (transceiving) way; should instead, in particular by configuration or constructive limits of the wireless identification electronic device 10, the step of transmission of the reply signal occur after the completion of the step of reception of the query signal, there will be a wireless identification electronic device 10 of type or with half duplex behaviour.

Preferably, but non-limiting thereto, the wireless identification electronic device 10 will be configured for receiving and transmitting signals with numerical modulation, for example FSK or ASK or PSK.

In an embodiment prior to the steps of the above-described method of reading of data, it can be provided the writing of data within the memory of the wireless identification electronic device 10. Subsequently, the method can comprise a step of configuration of the wireless identification electronic device 10 in such a way that this electronic device can be arranged in read only mode, so that the electronic data written in the memory can no longer be altered. The herein above-described steps may occur, depending on the cases:
before the wireless identification electronic device 10 is introduced in the elongated body 2 and/or in the collecting portion 3;
after the wireless identification electronic device 10 has been introduced, and in particular after the elongated body and/or the collecting portion 3 have been moulded, in particular co-moulded and/or injection moulded and/or overmoulded, in particular moulded by insertion or with core loss, in a mould wherein the wireless identification electronic device 10 was included.

The transmission of the reply signal implies that the latter passes through the wall of the container 20 and/or part of the elongated body 2 or of the collecting portion 3.

FIG. 8 shows a lateral view of an embodiment of the device 1 object of the present disclosure introduced within a container 20, in this case suitable for substantially containing the whole elongated body 2 and the collecting portion 3 within its length.

The container 20 presents substantially an axial development, a mouth in correspondence of a first end thereof and a portion of bottom in correspondence of a second end thereof opposed with respect to the first end.

In FIG. 8 is represented a particular embodiment of the device 1 that integrates a stopper for the container 20; the stopper, identified by the numerical reference 16, is arranged in correspondence of a portion of the elongated body 2 that is opposed with respect to the ending portion 2e thereof.

The stopper 16 is configured and specifically destined to be introduced at least partially inside the container 20, or to be screwed or interlocked on the container 20, to close it preferably hermetically, in a removable way.

In a non-limiting embodiment, the stopper 16 can be realized in plastic material. Alternatively the stopper 16 can be realized in rubber and have remarkable properties of elastic deformability to ensure the above-mentioned hermetic closing.

The stopper 16, if present, is joined to the elongated body 2 so as to remain unmovably joined to the latter, at least in operational configuration.

In FIG. 8 is it is visible a tag 12 having at least one alphanumeric and/or visual code; the alphanumeric and/or visual code represents at least part of the data stored in the memory of said wireless identification electronic device 10 and/or is destined, when electronically read, to address an electronic device 100 to the retrieval, in particular automated, of said data stored in the memory of said wireless identification electronic device 10 or to the retrieval of an electronic copy of said data stored in the memory of said wireless identification electronic device 10.

Preferably, but non-limiting thereto, the above-described tag 12 is positioned on the elongated body 2 or on a gripping and/or closing element 16 (the stopper) of the container 20 for the device 1.

In an optional embodiment, it is provided that the alphanumeric and/or visual code present on the tag comprises, or is, equivalent to the univocal identification code previously mentioned. In particular, if the tag 12 is positioned on the container 20 of the device 1, this allows to electronically and uniquely identify the pair device 1—container 20 and thus contributes to reduce the risk of unintended exchanges between devices 1 and containers 20 that may occur during the handling of a multiplicity of them.

From a manufacturing point of view, the wireless identification electronic device 10 is joined to the elongated body 2 and/or to the collecting portion 3 in the step of moulding, and preferably in said injection moulding and/or in said overmoulding, in particular in said moulding by insertion or with core loss.

In a preferred embodiment, the manufacturing process of the device 1 in fact provides to position the wireless identification electronic device 10 in correspondence of a predefined portion of a mould 200, that is subsequently closed to then realize the elongated body 2 and/or the collecting portion 3 (in particular of the supporting portion) by moulding, preferably by injection or co-moulding, in such a way that the plastics encloses at least partially, preferably integrally, the wireless identification electronic device 10.

In an embodiment, the elongated body 2 and/or the collecting portion 3 are overmoulded on the at least one first wireless identification electronic device 10, or at least partially around the at least one first wireless identification electronic device 10.

If the wireless identification electronic device 10 is introduced in a mould for the realization of the elongated body 2, and/or of the collecting portion 3 (in particular of the supporting portion), this wireless identification electronic device 10 is of heat-resistant type.

It is noted that the plastic materials that can be used for the manufacturing of the elongated body 2 and/or of the collecting portion 3, are characterized by melting temperatures that can also reach up to 320° C., and require moulding temperatures that, although they can start from 17-20° C., can also reach temperatures above 160° C.

For the purposes of the present disclosure, for wireless identification electronic device 10 is intended a device whose operating temperature reaches 320° C., or at least 300° C., or at least 250° C. or at least 200° C.

Alternatively or in combination with the previously mentioned properties of heat resistance, in a non-limiting embodiment the at least one wireless identification electronic device 10 can be of radiation-resistant type. For the purposes of the present disclosure, for radiation-resistant is intended a wireless identification electronic device 10 capable of resisting radiations of alpha and/or beta and/or gamma type, then radiation of ionizing type. Preferably the wireless identification electronic device 10 is capable of keeping unaltered the data contained in its memory for a predefined irradiation time with one or more of the above indicated radiations.

Thanks to this technical feature it is possible to keep unaltered the data contained in said memory, in particular at least the univocal identification code, in the sterilization of the device 1, that can occur—in addition to the thermal way—also by radiative way.

Finally, it is noted that if necessary the wireless identification electronic device 10 can be frost-resistant, and in particular be capable of withstanding without malfunctioning temperatures that can drop below −100° C., or below −150° C., reaching up to withstand temperatures substantially equal to −200° C. This can be useful for applications wherein it is necessary to submit the sample collected in the collecting portion 3 to very low temperatures, since the reaching of such temperatures does not impact on the functionality of the wireless identification electronic device 10; the traceability of the sample and of the device herein described is thus keepable.

The wireless identification electronic device 10 results thus irremovably fixed and enclosed, at least partially in the elongated body 2 and/or in the collecting portion 3. As previously described, it can be in the elongated body 2, in the collecting portion 3 or in an intermediate position between these two, but is arranged in a fixed position with respect to the latter.

The moulding process ensures a rapid, repeatable, cost-effective manufacturing for the device 1, despite the aggravation of costs inevitably present with respect to a traditional swab without the wireless identification electronic devices. The substantial inclusion of the wireless identification electronic device 10 within the elongated body 2, or within the collecting portion 3 or within an intermediate position between the last two, ensures that the wireless identification electronic device 10 is substantially irremovable, and this allows to ensure that if the data stored therein are not corrupted, there is no risk of data-sample exchange. The cataloging of samples, in association with devices 1 and relative data is thus ensured in terms of correctness.

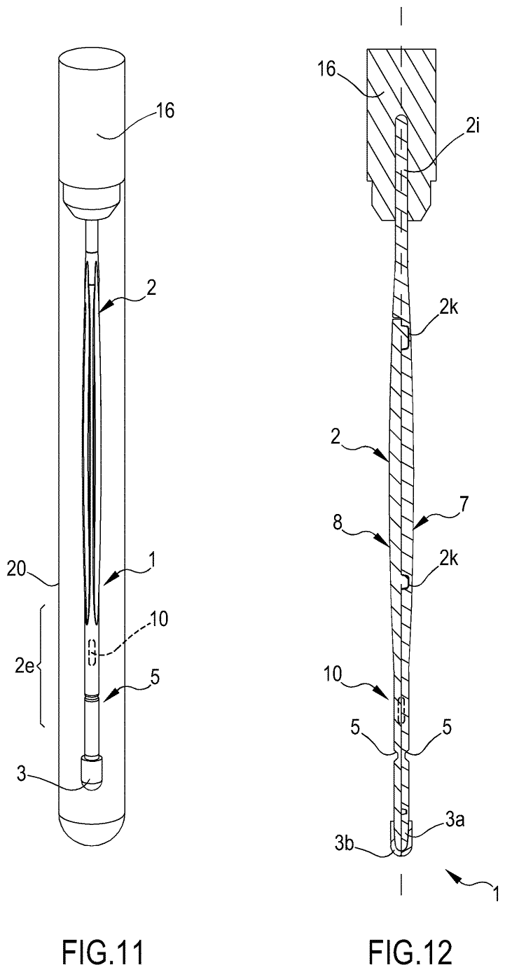

FIGS. 12, 13 and 14 show a particular embodiment of the device 1 object of the present disclosure wherein the elongated body is realized by a first half-body 7 and a second half-body 8. The elongated body can comprise a plurality of reinforcement ribs, arranged in the direction substantially parallel to the main development direction X (FIG. 11).

These first half-body 7 and second half-body 8 are different one another and selectively couplable, for defining an assembled configuration of the elongated body 2, and selectively separable the one from the other, for defining a disassembled condition of the elongated body 2.

The collecting portion 3 is realized and engaged both on a first end of the first half-body 7 and on a first end of the second half-body 8, and is selectively separable, after a separation of the first half-body 7 from the second half-body 8, for defining two half collecting portions respectively realized on the first half-body 7 and on the second half-body 8.

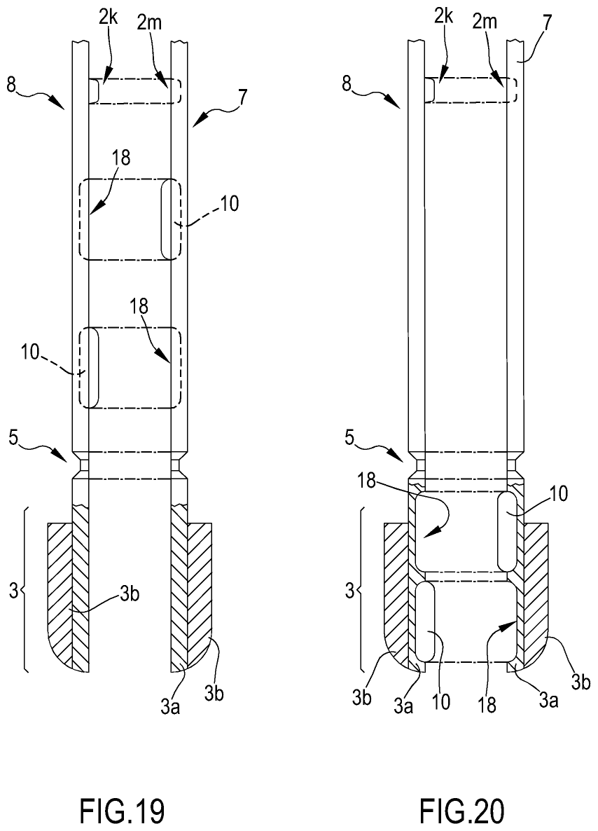

It is noted that if the device 1 is provided with a first and of a second wireless identification electronic device 10, it will be possible to have the following configurations:
said first wireless identification electronic device 10 is arranged in correspondence of the first half-body 7 and said second wireless identification electronic device 10 is arranged in correspondence of the second half-body 8 (FIG. 19), and/or
said first wireless identification electronic device 10 is arranged in correspondence of the half-collecting portion realized on the first half-body 7 and said second wireless identification electronic device 10 is arranged in correspondence of the half-collecting portion realized on the second half-body 8 (FIG. 20).

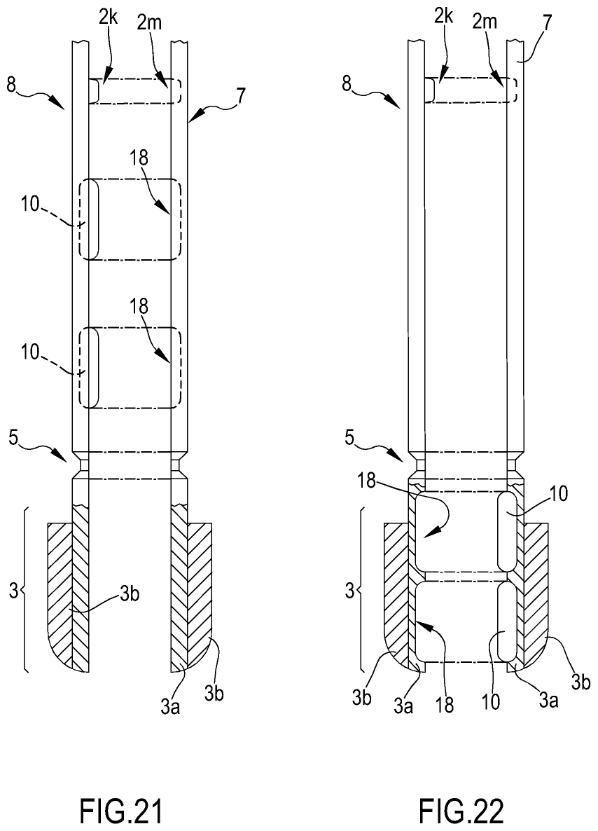

In a further embodiment said first and second wireless identification electronic device 10 are both arranged in correspondence of the second half-body 8 (FIG. 21) or, alternatively, are both arranged in correspondence of the first half-body 8 (solution not shown).

In even a further embodiment, said first and second wireless identification electronic device 10 are both arranged in correspondence of the half-collecting portion realized on the first half-body 7 (FIG. 22) or, alternatively, are both arranged in correspondence of the half-collecting portion realized on the second half-body 8 (solution not shown). Thanks to the above indicated technical features it is possible to divide the collected sample into the collecting portion in at least two portions and keep univocal track of them; both the sample of the first half-collecting portion and the sample of the second half-collecting portion can be tracked by a respective wireless identification electronic device 10.

From the functional point of view, the tracking data of the sample and/or of the device 1 and/or of analysis method of said sample and/or of collection date of the sample, and/or of transport conditions of the sample will be written in parallel (simultaneously or not) both on the first wireless identification electronic device 10 and on the second wireless identification electronic device 10, so that in both of them all the data necessary for the analysis and proper tracking of the samples can appear.

The univocal identification code of the first and of the second wireless identification electronic device 10 will be preferably distinct. In this way it will be possible to electronically distinguish the “first” part of the sample (first half-body 7) from the “second” part of the sample (second half-body 8), and then—also in absence of visible signs of difference between the first and the second half-body 7, 8, allow to distinguish which part of the sample is being considered. This is in particular useful if samples are treated with reagents, to avoid cross-contaminations.

The engagement between the first and the second half-body 7, 8 and their separation are possible by means of removable coupling means, that can be for example in form of engagement elements 2k.

In particular, the engagement elements 2k allow the support body 2 to operate between an assembled condition and a separation condition.

In the assembled condition, shown for example in FIGS. 9 and 10, the first and the second half-body 7, 8 are engaged between them; vice versa, in the separation condition, shown in FIG. 11, the first and the second half-body 7, 8 are separated.

The engagement elements 2k are preferably defined on the first and on the second half-body 7, 8 and can be for example of snap or interlocking or interference type. According to an embodiment shown in the attached figures, the engagement elements 2k comprise abutments and housing seats 2m for abutments 2k.

Preferably, the housing seats 2m are counter-shaped to the abutments 2k. As shown in the attached figures, the housing seats 2m can be definite on the first half-body 7 and the abutments 2k can be definite on the second half-body 8, or vice versa.

Alternatively, the first half-body 7 can comprise a or more housing seats 2m and a or more abutments 2k and the second half-body 8 may in turn comprise a or more abutments 2k and a or more housing seats 2m suitable for cooperating respectively with the housing seats 2m and with the abutments 2k of the first half-body 7. The abutments 2k and the housing seats 2m could also be alternated on the two half-bodies 7, 8, so that each of the two half-bodies 7, 8 presents both abutments 2k and housing seats 2m.

Preferably, the engagement elements are constituted by pairs of abutment-housing seat; in other words, to each abutment 2k corresponds a respective housing seat 2m. For example it is possible to realize an elongated body 2 provided with three pairs of abutment-housing seat.

The engagement elements (and in particular the pairs of abutment-housing seat) can be defined in correspondence of a plurality of sections of the first and/or of the second half-body 7, 8. For example, as shown in FIG. 12, the engagement elements can be defined in proximity of opposed ends of the first and/or of the second half-body 7, 8 (that is in proximity of the ends of the elongated body 2) and in correspondence of an intermediate portion of the first and/or of the second half-body 7, 8. The embodiment of the device 1 herein described is represented in a perspective way in FIG. 11 within a container 20, the features of which have already been previously shown.

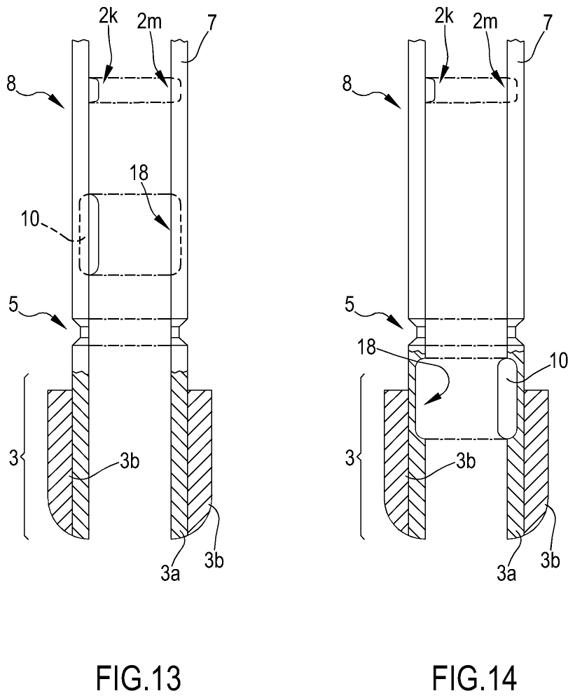

As represented schematically in FIG. 13, in an embodiment the wireless identification electronic device 10 is partially embedded within the second half-body of the elongated body 2; in the first half-body 7 there is a recess 18 suitable for housing the portion of the wireless identification electronic device 10 not contained in the second half-body 8.

Clearly, if the size of the wireless identification electronic device 10 so allows, it will be possible to keep the whole wireless identification electronic device 10 within the second half-body of the elongated body 2; in this case it will not be necessary the presence of the recess 18. It is intended that the position of the wireless identification electronic device 10 can be exchanged at the manufacturing level between the first half-body 7 and the second half-body 8. For this reason it is intended that the recess 18 can be present on the second half-body of the elongated body 2 if the wireless identification electronic device 10 is partially included in the first half-body of the elongated body 2.

In this last case the manufacturing process can comprise the introduction of the wireless identification electronic device 10 in the mould 200 and the realization of the first (or of the second) half-body 7, 8 with the relative supporting portion 3a of the collecting portion 3.

A successive step can comprise the manufacturing of the portion of elongated body 2 and of relative supporting portion, that does not comprise the wireless identification electronic device 10.

The present disclosure refers also to a method of manufacturing a device 1 for the collection and the transfer of samples, preferably samples of biological material.

FIG. 13 shows an embodiment of the device 1 wherein a first wireless identification electronic device 10 is partially embedded within the second half-body of the body 2; in the first half-body 7 there is a recess 18 suitable for housing the portion of the first wireless identification electronic device 10 not contained in the second half-body 8.

Alternatively, as shown in FIG. 14, a second wireless identification electronic device 10 can be partially embedded within the first half-body 7 of the body 2; in the second half-body 8 there is a recess 18 suitable for housing the portion of the second wireless identification electronic device 10 not contained in the first half-body 7.

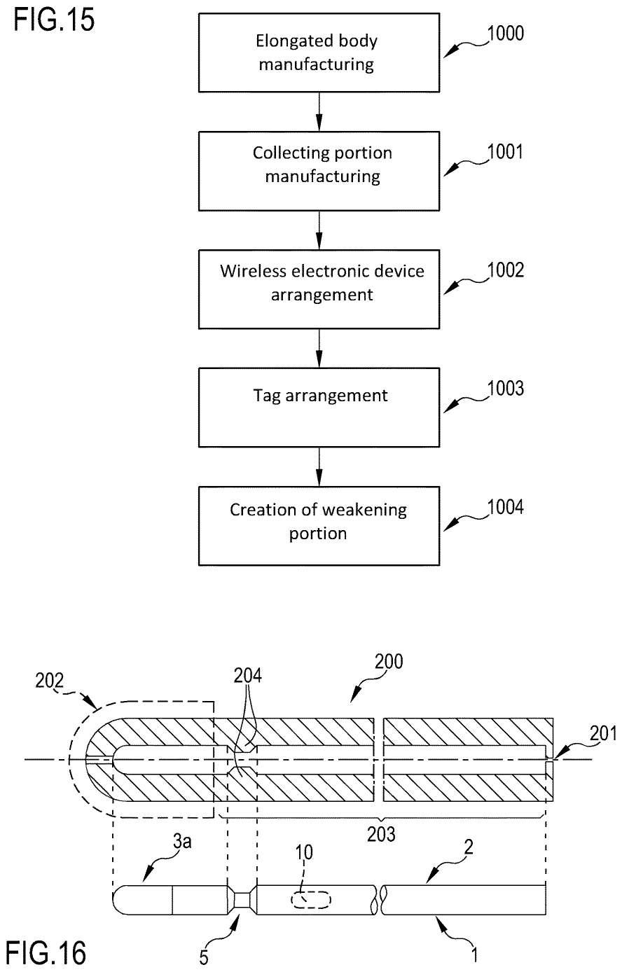

With reference to the schematically represented steps in FIG. 15, the method herein described first of all comprises a step of manufacturing 1000 an elongated body 2 extending at least partially along a main development direction X substantially axial and comprising at least one gripping portion 2i destined to be grasped by a user and at least one ending portion 2e.

Should the elongated body 2 be realized by a first half-body and by a second half-body 7, 8, clearly the step of manufacturing 1000 will comprise the realization of the first half-body 7 and the realization of the second half-body 8.

The method object of the present disclosure also comprises a step of manufacturing 1001 a collecting portion 3 configured for collecting and/or absorbing and/or retaining and/or releasing a quantity of a sample, preferably a sample of biological material, the step of manufacturing 1001 comprising an arrangement of the collecting portion 3 in correspondence of said ending portion 2e of the elongated body 2.

The step of manufacturing 1000 the elongated body 2 and the step of manufacturing 1001 the collecting portion 3 can take place substantially simultaneously, being of in fact a single step of a manufacturing process of a body seamlessly identified.

The method object of the present disclosure also comprises a step of positioning 1002 of a wireless identification electronic device 10 configured for receiving and/or transmitting and/or storing electronic data on a wireless communication channel.

The step of positioning 1002 comprises an arrangement of said wireless identification electronic device 10 at least partially within said elongated body 2 and/or within said collecting portion 3. Also the step of positioning 1002 can occur in substantial temporal simultaneity with the steps indicated by the references 1000 and 1001.

In an embodiment the step of positioning 1002 is a step that, in the manufacturing process, occurs at the temporal level contextually to the step of manufacturing 1000 the elongated body 2 and/or to the step of manufacturing 1001 the collecting portion 3.

More in particular, the step of manufacturing 1000 the elongated body 2 and/or the step of manufacturing 1001 the collecting portion 3 comprise a step of moulding, preferably a step of injection moulding and/or of overmoulding, in particular moulding by insertion or with core loss, and/or co-moulding, of said elongated body 2 and/or said collecting portion 3.

Following of the steps of manufacturing 1000, 1001 the elongated body 2 and/or of the collecting portion 3, and with the completion of the step of positioning 1002, the wireless identification electronic device 10 is joined to the elongated body 2 and/or to the collecting portion 3 in said step of moulding, and preferably in the step of injection moulding and/or in said step of overmoulding, in particular moulding by insertion or with core loss.

The assembly realized by the steps of manufacturing 1000, 1001 the elongated body 2 and/or of the collecting portion 3 and by the step of positioning 1002 determines an immovable fastening of the wireless identification electronic device 10 to the elongated body 2 and/or to the collecting portion 3.

In a specific and not-limiting embodiment, the step of positioning 1002 comprises a positioning of the wireless identification electronic device 10 in a position (within the mould 200) such that, after the step of manufacturing 1000 the elongated body and/or after the step of manufacturing 1001 the collecting portion 3, said wireless identification electronic device 10 is integrally within the elongated body 2 and/or within said collecting portion 3.

FIG. 16 shows in particular a solution wherein the wireless identification electronic device 10 is in a position wherein, downwards of the injection of the plastic material within the mould 200, this device will be integrally within the elongated body 2. The injection occurs in correspondence of an inlet hole 201 of the mould 200, preferably positioned in a rear end of the mould itself.

In FIG. 16 are identified a first portion of the mold 200, identified by the numerical reference 202, that roughly corresponds to the supporting portion 3a of the collecting portion, and a second portion 203 of the mould 200, that roughly corresponds to the portion of the elongated body 2.

After the step of positioning 1002, of the step of manufacturing 1000 the elongated body 2 and of the step of manufacturing 1001 the collecting portion 3, said elongated body 2 and/or said collecting portion 3 defines (or define) the covering layer 4 of the wireless identification electronic device 10.

Depending on the fact that the collecting portion 3 is realized seamlessly with the body 2 or not, the step of manufacturing 1000 the elongated body 2 and the step of manufacturing 1001 the collecting portion 3 determine a conjunction of the collecting portion 3 a said elongated body 2 and/or determine an integral and/or seamless realization between the collecting portion 3 and the elongated body 2.

Getting into more detail, the step of manufacturing 1001 the collecting portion 3 comprises:
a first sub-step of manufacturing a supporting portion 3a for the swab 3b;
a second sub-step of manufacturing a swab 3b having one among the following properties: hydrophilic, hydrophobic or neutral.

Preferably the second sub-step of manufacturing the swab 3b is a step of manufacturing a swab 3b that comprises a plurality of fibers provided with hydrophilic capacity, and optionally is a step of flocking, and/or of fiber winding and/or of arrangement of a foam.

It is noted in particular that is the first sub-step of manufacturing the supporting portion 3a a can simultaneously occur at the step of manufacturing the elongated body 2, both in an embodiment in single piece and in an embodiment wherein this elongated body is realized through at least one first portion 7 and a second portion.

The step of positioning 1002 can comprise a positioning of said wireless identification electronic device 10 within said supporting portion 3a. In this case, as previously described, said covering layer 4 becomes part of the supporting portion 3a.

In a particular embodiment, the step of positioning 1002 determines, following the completion at least of the step of manufacturing 1000 the elongated body 2, the positioning of the outer surface 10x of the wireless identification electronic device 10 in substantial contact with the outer surface 2x of the elongated body 2 or the positioning of the outer surface 10x of the wireless identification electronic device 10 in such a way that at least part of said outer surface 10x lies in correspondence of said outer surface 2x of the elongated body 2 and/or protrudes partially outside elongated body 2.

Optionally, the step of positioning 1002 determines the positioning of the wireless identification electronic device 10 in substantial correspondence of said ending portion 2e or in correspondence of said gripping portion 2i.

In a non-limiting embodiment, the method comprises a step 1004 of realization of a weakening portion 5 on the elongated body 2. Preferably, but non-limiting thereto, the step 1004 comprises the reduction of the transversal section of a portion of the elongated body 2 after at least the step of manufacturing 1000 the elongated body 2, or—alternatively—comprises the use of a mould 200 that in correspondence of the area wherein it has to be the weakening portion 5, has an abutment 204 facing toward the inside of the cavity identified thereby, so as to determine the above-mentioned reduction of the transversal section. Following the completion of the step 1004, the weakening portion 5 lies in a translated position, along the direction X of maximum extension of the elongated body 2, with respect to the position in correspondence of which it is positioned the wireless identification electronic device 10.

In a specific embodiment, the step of realization 1000 of the body determines, or implies, the realization of the step 1004, that then occurs in substantial temporal simultaneity with the step 1000 of manufacturing the elongated body 2.

As already previously anticipated, the step of realization 1000 of the elongated body 2 can comprise a step of realization of a first half-body 7 and of a second half-body 8 different one another and selectively couplable, for defining an assembled configuration of the elongated body 2, and selectively separable the one from the other, for defining a disassembled condition of the elongated body 2.

The step of realization 1001 of the collecting portion 3 comprises a realization and an engagement of the collecting portion 3 both on a first end of the first half-body 7 and on a first end of the second half-body 8, in such a way that said collecting portion 3 is selectively separable, after a separation of the first half-body 7 from the second half-body 8, for defining two half collecting portions 3a respectively realized on the first half-body 7 and on the second half-body 8.

It is in particular noted that in an embodiment, the assembly of the step of positioning 1002 and of the step of manufacturing 1000 the body (2) and/or of manufacturing 1001 the collecting portion 3, determines the arrangement of the first wireless identification electronic device 10 in correspondence of said first half-body 7 and of the second wireless identification electronic device 10 in correspondence of the second half-body 8, and/or determines the arrangement of the first wireless identification electronic device 10 in correspondence of the half-collecting portion realized on the first half-body 7 and of the second wireless identification electronic device 10 in correspondence of the half-collecting portion realized on the second half-body 8.

Preferably, but non-limiting thereto, the step of positioning 1002 of the wireless identification electronic device 10 is a step of positioning of a wireless identification electronic device 10 of substantially three-dimensional shape such that a main extension axis of the wireless identification electronic device 10 results aligned to the main development direction X of the elongated body 2.

In a non-limiting embodiment, the method provides a step 1003 of arrangement of at least one tag 12 on said device 1, said tag 12 having at least one alphanumeric and/or visual code.

Preferably, the alphanumeric and/or visual code represents at least part of the data stored in the memory of said wireless identification electronic device 10 and/or is destined, when electronically read, ad address the reader device 100 to the retrieval, in particular automated, of said data stored in the memory of said wireless identification electronic device 10 or to the retrieval of an electronic copy of said data stored in the memory of said wireless identification electronic device 10.

Although this is not a limitation, the step 1003 of arrangement of at least one tag 12 on said device 1 comprises a positioning of said tag 12 on the elongated body 2 or on a gripping and/or closing element of the container 20.

It is noted finally that in a further embodiment of the method herein described, the step of positioning 1002 is a step of positioning of the wireless identification electronic device 10 in correspondence of a recess obtained in a portion of the elongated body 2 and/or in a recess obtained in a sub-portion of the collecting portion 3, and in particular in a sub-portion of the supporting portion 3a.

In particular, the recess can be obtained during the, or downwards of the, step of manufacturing the elongated body 2 and/or of the collecting portion 3, in particular of the supporting portion 3a.

In this case, the step of positioning 1002 of the wireless identification electronic device 10 comprises or is followed by, a step of gluing of the wireless identification device 10 in the above-mentioned recess and/or by a step of insertion by contrast of the wireless identification device 10 in the above-mentioned recess.

The present embodiment of the step of positioning 1002 can be convenient in case of presence of a first half-body 7 and of a second half-body 8 as previously described.

Therefore, it is clear that if there are a first half-body 7 and a second half-body 8, the at least one wireless identification electronic device 10 can be included at least partially in the first half-body 7 and/or in the second half-body 8 by means of a process of moulding or by a process of gluing and/or insertion by contrast successive to the step of manufacturing, also by moulding, the first half-body 7 and/or of the second half-body 8.

The Applicant notes that when the wireless identification electronic device 10 is included in the elongated body 2 and/or in the collecting portion 3 in a step of moulding wherein it is introduced in the mould 200, it is determined a structural difference in the device 1 that makes it distinguishable from other wherein the wireless identification electronic device 10 is included in the elongated body 2 and/or in the collecting portion 3 by means of gluing and/or of insertion by contrast. In particular, by sectioning the device 1 in correspondence of the wireless identification electronic device 10 is easily identifiable by which specific step this latter device has been fixed.

The present disclosure also concerns an equipment for the manufacturing of a device 1 for the collection and the transfer of samples of biological, microbiological or chemical material according to what is herein described. In particular, the equipment herein described is destined—when activated—to execute a method of manufacturing of a device 1 for the collection and the transfer of samples of biological, microbiological or chemical material that mainly comprises:
a step of manufacturing (block 1000) a body 2, preferably elongated and extending at least partially along a main development direction X substantially axial, comprising at least one gripping portion 2i destined to be grasped by a user and at least one ending portion 2e;
a step of manufacturing (block 1001) a collecting portion 3 configured for collecting and/or absorbing and/or retaining and/or releasing a quantity of a sample of biological, microbiological or chemical material, the step of manufacturing 1001 comprising preferably an arrangement of the collecting portion 3 in correspondence of said ending portion 2e,
a step of positioning (block 1002) of a wireless identification electronic device 10 configured for receiving and/or transmitting and/or storing electronic data on a wireless communication channel, the step of positioning 1002 comprising an arrangement of said wireless identification electronic device 10 at least partially within said body 2 and/or within said collecting portion 3.

Preferably, but non-limiting thereto, the equipment herein described is configured and specifically destined to be activated in a substantially automated way, in order to manufacture a large quantity of devices 1 without substantial intervention by the user.

The equipment herein described can comprise a or more stations, which can be next between them or substantially separates. Conveyors or conveyor belts or pliers or pushers can push one or more semi-processed devices 1 from one station to another. In particular, stations distinct between them can be present for the realization of the swab 3b and of the supporting portion 3a therefor.

At least one of said stations is a station comprising the mould 200. At least one of said stations is a station specifically configured for realizing a flocking of synthetic fibers on a support.

The equipment herein described is conveniently provided with a control unit which can comprise a or more processors of general-purpose type, on which it is executed a computer program, and/or can comprise a or more processors of application specific type, and/or can comprise FPGA or PLC or further controllers for servo actuators necessary for the realization of the steps of the previously described method.

If the control unit shall use a software program, it appears clear that the above-mentioned software program will comprise portions of software code that when executed are destined to cause the previously described steps, and any further step of processing directly inferable from the present description.

Regarding specifically the reader device 100, it is noted that in an embodiment this reader device 100 can assume shape of portable device (handheld), configured for being carried by hand by the operator. In an alternative embodiment, represented schematically in FIG. 18, this reader device 100 can be a fixed device and/or a module of a more complex machine and integrate an antenna 101 for the reading and the transmission of signals respectively from and/or towards devices 1 as the ones herein described, and a or more housings 102 suitable for housing containers 20 with, if necessary, included with devices 1, in a predetermined spatial configuration, for example a configuration in which the previously described axes X assume substantially vertical spatial orientation.

The invention is not limited to the embodiments shown in the attached figures; for this reason, reference numbers which are in the claims that following are provided for the sole purpose of increasing the intelligibility thereof and shall not be intended as limiting.

It is then clear that to the object of the present disclosure can be applied additions, modifications or variants obvious for an expert in the art without exiting from the scope of the attached claims.

1. A device for the collection and the transfer of samples of biological, microbiological or chemical material, comprising:
a body, preferably elongated and extending at least partially along a main development direction substantially axial, comprising at least one gripping portion destined to be grasped by a user and at least one ending portion;
a collecting portion, preferably arranged in correspondence of said ending portion, the collecting portion being configured for collecting and/or absorbing and/or retaining and/or releasing a quantity of a sample of biological, microbiological or chemical material; and
at least one wireless identification electronic device, configured for receiving and/or transmitting and/or storing electronic data on a wireless communication channel, the wireless identification electronic device being arranged, at least partially, within said body and/or within said collecting portion.

2. The device according to claim 1, wherein at least one between said body and said collecting portion is realized by moulding, preferably injection moulded and/or overmoulded, in particular moulded by insertion or with core loss, and/or co-moulding,
and wherein said at least one wireless identification electronic device is joined to said body and/or to said collecting portion in said moulding, and preferably in said injection moulding and/or in said overmoulding, in particular by insertion or with core loss, and/or in said co-moulding, being introduced within a mould;
or wherein said at least one wireless identification electronic device is joined to said body and/or to said collecting portion by gluing and/or insertion by contrast, optionally carried out after said moulding;
said at least one wireless identification electronic device being irremovably fixed to said body and/or to said collecting portion.

3. The device according to claim 1, wherein said at least one wireless identification electronic device is positioned and enclosed integrally within said body and/or within said collecting portion, and/or wherein said body and/or said collecting portion defines a covering layer of said at least one wireless identification electronic device,
optionally said covering layer extending around said at least one wireless identification electronic device in a plurality of radial directions, extending in a direction oblique, preferably orthogonal, to the main development direction of said body.

4. The device according to claim 3, wherein said collecting portion is joined to said body and/or is realized integrally and/or seamlessly with said body,
said collecting portion comprising:
a swab having one among the following properties: hydrophilic, hydrophobic or neutral;
preferably comprising a plurality of fibers provided with hydrophilic capacity, and optionally being a flocked swab, and/or with wound fiber, and/or in foam;
a supporting portion for said swab;
preferably wherein said at least one wireless identification electronic device is positioned within said supporting portion;
wherein said covering layer is part of said supporting portion;
and wherein said at least one wireless identification electronic device comprises a respective outer surface, and said body comprises a respective outer surface,
and wherein the outer surface of said at least one wireless identification electronic device is in substantial contact with the outer surface of the body or faces on said outer surface of the body and/or protrudes partially outside said body,
optionally wherein said at least one wireless identification electronic device is positioned in substantial correspondence of said ending portion or in correspondence of said gripping portion.

5. (canceled)

6. The device according to claim 1, comprising at least one weakening portion; said weakening portion being positioned on said body and being configured for facilitating a separation between at least part of the body and the collecting portion,
and/or wherein the body comprises at least one first half-body and a second half-body different one another and selectively couplable, for defining a assembled configuration of the body, and selectively separable the one from the other, for defining a disassembled condition of the body, and wherein the collecting portion is realized and engaged both on a first end of the first half-body and on a first end of the second half-body, and is selectively separable, after a separation of the first half-body from the second half-body, for defining two half collecting portions respectively realized on the first half-body and on the second half-body,
said at least one wireless identification electronic device being arranged at least partially in said first half-body or in correspondence of the collecting portion, preferably of the supporting portion, joined to said first half-body and/or realized integrally and/or seamlessly with said first half-body,
or being arranged at least partially in said second half-body or in correspondence of the collecting portion, preferably of the supporting portion, joined to said first half-body and/or realized integrally and/or seamlessly with said first half-body.

7. The device according to claim 1, wherein said at least one wireless identification electronic device is in substantially three-dimensional shape, preferably having its own main extension axis, substantially parallel to said main development direction of the body, and/or wherein said at least one wireless identification electronic device is of passive type, or of active type, or of semi-active type, or of semi-passive type;
and/or wherein said at least one wireless identification electronic device comprises at least one memory configured for storing, in use, tracking data of said sample and/or of the device and/or of analysis method of said sample and/or of collection date of said sample, and/or of transport conditions of said sample;
and/or wherein said at least one wireless identification electronic device is of read only type, or is configured for operating in read only mode, in such a way that the data written in said memory can be only read, or wherein said at least one wireless identification electronic device is of read-write type, or is configured for operating in read-write mode, in such a way that at least part of the data written in said memory can be read and written.

8. The device according to claim 1, comprising at least one tag having at least one alphanumeric and/or visual code; said alphanumeric and/or visual code representing at least part of the data stored in the memory of said at least one wireless identification electronic device and/or being destined to, when electronically read, address a reader device to the retrieval, in particular automated, of said data stored in the memory of said at least one wireless identification electronic device or to the retrieval of an electronic copy of said data stored in the memory of said at least one wireless identification electronic device;
said tag being preferably positioned on the body or on a gripping and/or closing element of a container for said device, fixed to said body in correspondence of said gripping portion or of a portion of said body opposed with respect to said ending portion.

9. The device according to claim 1, comprising at least:
a first wireless identification electronic device and at least one second wireless identification electronic device arranged at least partially within said first body; or
a first wireless identification electronic device arranged at least partially within said first body and at least one second wireless identification electronic device arranged within said collecting portion;
a first wireless identification electronic device and at least one second wireless identification electronic device arranged within said collecting portion;
a first wireless identification electronic device arranged within said collecting portion and at least one second wireless identification electronic device arranged at least partially within said first body.

10. The device according to claim 6, comprising at least one second wireless identification electronic device, configured for receiving and/or transmitting and/or storing electronic data on a wireless communication channel, the wireless identification electronic device being arranged, at least partially, within said body and/or within said collecting portion,
and wherein said first wireless identification electronic device is arranged in correspondence of said first half-body and said second wireless identification electronic device is arranged in correspondence of said second half-body, or
said first wireless identification electronic device is arranged in correspondence of the half-collecting portion realized on the first half-body and said second wireless identification electronic device is arranged in correspondence of the half-collecting portion realized on the second half-body, or
said first wireless identification electronic device is arranged in correspondence of the first half-body and said second wireless identification electronic device is arranged in correspondence of the half-collecting portion realized on the second half-body.

11. A method of manufacturing of of claim 1, the method comprising:
a step of manufacturing a body, preferably elongated and extending at least partially along a main development direction substantially axial, comprising at least one gripping portion destined to be grasped by a user and at least one ending portion;
a step of manufacturing a collecting portion configured for collecting and/or absorbing and/or retaining and/or releasing a quantity of a sample of biological, microbiological or chemical material, the step of manufacturing comprising preferably an arrangement of the collecting portion in correspondence of said ending portion,
a step of positioning of at least one wireless identification electronic device configured for receiving and/or transmitting electronic data on a wireless communication channel, the step of positioning comprising an arrangement of said at least one wireless identification electronic device at least partially within said body and/or within said collecting portion.

12. The method according to claim 11, wherein the step of manufacturing the body and/or the step of manufacturing a collecting portion comprises a step of moulding, preferably a step of injection moulding and/or of overmoulding, in particular moulding by insertion or with core loss, and/or co-moulding, of said body and/or said collecting portion,
and wherein said at least one wireless identification electronic device is joined to said body and/or to said collecting portion in said step of moulding, and preferably in said step of injection moulding and/or in said step of overmoulding, in particular of moulding by insertion or with core loss, and/or co-moulding, being introduced within a mould;
or wherein the step of manufacturing the body and/or the step of manufacturing a collecting portion comprises a step of moulding, preferably a step of injection moulding and/or of moulding by insertion and/or co-moulding, of said body and/or said collecting portion, and said at least one wireless identification electronic device is joined to said body and/or to said collecting portion by gluing and/or insertion by contrast, optionally carried out after said moulding;
the assembly realized by the steps of manufacturing the body and/or the collecting portion and by the step of positioning determining an immovable fastening of said at least one wireless identification electronic device to said body and/or to said collecting portion.

13. The method according to claim 11, wherein the step of positioning comprises a positioning of said at least one wireless identification electronic device in a position such that, after the step of manufacturing the body and/or after the step of manufacturing the collecting portion, said at least one wireless identification electronic device is integrally within said body and/or within said collecting portion, and wherein after the step of positioning, of the step of manufacturing the body and of the step of manufacturing the collecting portion, said body and/or said collecting portion defines a covering layer of said at least one wireless identification electronic device,
optionally said covering layer extending around said at least one wireless identification electronic device in a plurality of radial directions, extending in a direction oblique, preferably orthogonal, to the main development direction of said body;
wherein the step of manufacturing the body and the step of manufacturing the collecting portion determine a conjunction of the collecting portion to said body and/or determine an integral and/or seamless realization between the collecting portion and the body,
the step of manufacturing the collecting portion comprising:
a first sub-step of manufacturing a supporting portion for said swab;
a second sub-step of manufacturing a swab having one among the following properties: hydrophilic. hydrophobic or neutral, preferably comprising a plurality of fibers provided with hydrophilic capacity, and optionally being a step of flocking, and/or of fiber winding and/or of arrangement of a foam;
preferably the step of positioning comprising a positioning of said at least one wireless identification electronic device within said supporting portion;
said covering layer being part of said supporting portion.

14. (canceled)

15. The method according to claim 11, wherein:
said at least one wireless identification electronic device comprises a respective outer surface,
said body presents a respective outer surface,
said step of positioning determines, following the completion at least of the step of manufacturing the body, the positioning of the outer surface of said at least one wireless identification electronic device in substantial contact with the outer surface of the body or the positioning of the outer surface of said at least one wireless identification electronic device in such a way that at least part of said outer surface lies in correspondence of said outer surface of the body and/or protrudes partially outside said body,
optionally wherein said step of positioning determines the positioning of said at least one wireless identification electronic device in substantial correspondence of said ending portion or in correspondence of said gripping portion.

16. The method according to claim 11, comprising a step of realization of a weakening portion on said body, the weakening portion being configured for facilitating a separation between at least part of the body and the collecting portion,
and/or wherein the step of manufacturing the body comprises a step of realization of a first half-body and of a second half-body different one another and selectively couplable, for defining a assembled configuration of the body, and selectively separable the one from the other, for defining a disassembled condition of the body, and wherein the step of realization of the collecting portion comprises a realization and an engagement of the collecting portion both on a first end of the first half-body and on a first end of the second half-body, in such a way that said collecting portion is selectively separable, after a separation of the first half-body from the second half-body, for defining two half collecting portions respectively realized on the first half-body and on the second half-body,
the assembly of the step of positioning and of the step of manufacturing the body and/or of realization of the collecting portion, determining the arrangement of said at least one wireless identification electronic device at least partially in said first half-body or in correspondence of the collecting portion, preferably of the supporting portion, joined to said first half-body and/or realized integrally and/or seamlessly with said first half-body,
or determining the arrangement of the wireless identification electronic device at least partially in said second half-body or in correspondence of the collecting portion, preferably of the supporting portion, joined to said first half-body and/or realized integrally and/or seamlessly with said first half-body.

17. The method according to claim 11, wherein the step of positioning of said at least one at least one wireless identification electronic device is a step of positioning of a wireless identification electronic device of substantially three-dimensional shape such that a main extension axis of said at least one wireless identification electronic device results aligned to the main development direction of the body,
wherein said at least one wireless identification electronic device is of passive type, or of active type, or of semi-active type or of semi-passive type; the step of positioning being specifically destined to allow a storage, in a memory of said at least one wireless identification electronic device, of tracking data of said sample and/or of the device and/or of analysis method of said sample and/or of collection date of said sample, and/or of transport conditions of said sample,
and/or wherein said at least one wireless identification electronic device is of read only type, or is configured for operating in read only mode, in such a way that the data written in said memory can be only read, or wherein said at least one wireless identification electronic device is of read-write type, or is configured for operating in read-write mode, in such a way that the data written in said memory can be read and written.

18. The method according to claim 11, comprising a step of arrangement of at least one tag on said device, said tag having at least one alphanumeric and/or visual code; said alphanumeric and/or visual code representing at least part of the data stored in the memory of said at least one wireless identification electronic device and/or being destined, when electronically read, to address a reader device to the retrieval, in particular automated, of said data stored in the memory of said at least one wireless identification electronic device or to the retrieval of an electronic copy of said data stored in the memory of said at least one wireless identification electronic device,
preferably the step of arrangement of at least one tag on said device comprising a positioning of said tag on the body or on a gripping and/or closing element of a container for said device, fixed to said body in correspondence of said gripping portion or of a portion of said body opposed with respect to said ending portion.

19. The method according to claim 11, wherein the step of positioning comprises:
an arrangement of a first wireless identification electronic device and of at least one second wireless identification electronic device at least partially within said first body; or
an arrangement of a first wireless identification electronic device at least partially within said first body and an arrangement of at least one second wireless identification electronic device within said collecting portion;
an arrangement of a first wireless identification electronic device and of at least one second wireless identification electronic device within said collecting portion;
an arrangement of a first wireless identification electronic device within said collecting portion and of at least one second wireless identification electronic device at least partially within said first body.

20. The method according to claim 16, comprising a step of positioning of at least one second wireless identification electronic device configured for receiving and/or transmitting and/or storing electronic data on a wireless communication channel, the step of positioning comprising an arrangement of said at least one wireless identification electronic device at least partially within said body and/or within said collecting portion;
and wherein the assembly of the step of positioning (1002) and of the step of manufacturing the body and/or of manufacturing (1001) the collecting portion, determines the arrangement of the first wireless identification electronic device in correspondence of said first half-body and of the second wireless identification electronic device in correspondence of the second half-body, or
determines the arrangement of the first wireless identification electronic device in correspondence of the half-collecting portion realized on the first half-body and of the second wireless identification electronic device in correspondence of the half-collecting portion realized on the second half-body, or
determines the arrangement of the first wireless identification electronic device in correspondence of the first half-body and of the second wireless identification electronic device in correspondence of the half-collecting portion realized on the second half-body.

21. A method of electronic reading of electronic data written in wireless identification electronic device contained in a device for the collection and the transfer of samples according to claim 1, the method comprising:
a step of positioning of the wireless identification electronic device in proximity of a reader device;
a step of transmission of a predefined query signal from the reader device towards the wireless identification electronic device;
a step of reception of the query signal, the reception occurring on said at least one wireless identification electronic device, the step of reception determining the electronic access to a memory of said at least one wireless identification electronic device and the loading of at least part of the data contained in said memory, for a successive retransmission of a reply signal;
a step of transmission of the reply signal towards the reader device, said reply signal containing at least part of the data stored in said memory.

22. The method according to claim 21, wherein said loading is a loading electronically carried out in an automatic way by a control unit of said at least one wireless identification electronic device and/or wherein the access to the memory is automatically carried out upon reception of the query signal and/or wherein the step of transmission occurs in an automatic way after the electronic access to the memory.

23. (canceled)

24. (canceled)

25. (canceled)
